(19) United States
(12) Patent Application Publication
(10) Pub. No.: US 2024/0174752 A1
(43) Pub. Date: May 30, 2024

(54) MODULATING PRODUCT QUALITY OF ASYMMETRIC MULTISPECIFIC ANTIBODIES THROUGH THE USE OF TEMPERATURE

(71) Applicant: AMGEN INC.

(72) Inventors: Li ZHANG, Camarillo, CA (US); Hedieh BARKHORDARIAN, Newbury Park, CA (US); Ewelina ZASADZINSKA, Thousand Oaks, CA (US); Jonathan DIEP, Thousand Oaks, CA (US)

(73) Assignee: AMGEN INC., Thousand Oaks, CA (US)

(21) Appl. No.: 18/284,590

(22) PCT Filed: Apr. 26, 2022
(86) PCT No.: PCT/US22/26261

§ 371 Date: Sep. 28, 2023

(51) Int. Cl. C07K 16/28; C07K 14/54

(52) U.S. Cl. CPC C07K 16/2818; C07K 14/54; C07K 2317/31; C07K 2319/30

(57) ABSTRACT

The present invention relates to the field of biopharmaceutical manufacturing. In particular, the invention relates to using temperature as a lever to modulate product quality during upstream operations.

This application claims the benefit of U.S. Provisional Application No. 63/180,220, filed Apr. 27, 2021, which is hereby incorporated by reference in its entirety and for all purposes as if fully set forth herein.

### SEQUENCE LISTING

Incorporated by reference in its entirety is a computer-readable nucleotide/amino acid sequence listing submitted concurrently herewith and identified as follows: 792,009 byte ASCII (Text) file named “A-2777-WO01-SEC_SeqListing.txt”; created on Mar. 8, 2022.

### FIELD OF DISCLOSURE

The present invention relates to the field of biopharmaceutical manufacturing. In particular, the invention relates to using temperature as a lever to modulate product quality during manufacturing operations, particularly during upstream cell culture operations.

### BACKGROUND

Monoclonal antibody biotherapeutics are the largest sector of the biopharmaceuticals market. However, because monoclonal antibodies can only bind to a single target, they are limited in their effectiveness as many diseases are multifactorial. Engineered multispecific antibodies are being developed to meet these challenges. These multispecific antibodies can have any number of unique peptide sequences and can be designed with multi-target affinity. The unique properties of these proteins offer improvements over traditional monoclonal antibody therapeutics and are proving to be effective next-generation biotherapeutics that can take advantage of an enormous variety of formats to meet even more challenging therapeutic indications.

However, there is not much information related to manufacturing multispecific antibodies, particularly those that are asymmetric in structure, so platforms and processes developed for monoclonal antibodies are often applied with varying degrees of success. Asymmetric multispecific antibodies are highly engineered and subjecting such proteins to upstream and downstream manufacturing processes under conditions typical for monoclonal antibodies can influence the product quality of the expressed and/or purified proteins. For example, production of product-related impurities during cell culture can complicate downstream purification operations and lower the product quality of the desired asymmetric multispecific drug product. It would therefore be beneficial to optimize conditions during upstream manufacturing operations of asymmetric multispecific antibodies to lessen any negative impact on product quality.

The invention described herein meets this need by making use of temperature as a lever during cell line development and cell culture operations to improve the product quality of recombinant asymmetric multispecific antibody drug substances.

### BRIEF SUMMARY OF THE INVENTION

The invention provides a method for modulating the product quality of a recombinant asymmetric multispecific antibody expressed by a cell during cell culture comprising the steps: (a) establishing at least two cell cultures each inoculated with the same cell line expressing the asymmetric multispecific antibody; (b) culturing at least one cell culture at a first temperature regime that consists of a single temperature and at least one cell culture at a second temperature regime; (c) comparing at least one product-related impurity in the cell culture at each temperature regime; (d) selecting the temperature regime that reduces the expression of at least one product-related impurity comprising an unpaired or mis-paired long heavy chain; (e) culturing the cell line at the selected temperature regime; and (f) harvesting the recombinant asymmetric multispecific antibody. In one embodiment the first temperature regime is selected from 36° C. to 37° C. In one embodiment the second temperature regime comprises a temperature shift from a first temperature to a second temperature that is higher or lower than the first temperature. In a related embodiment the second temperature is selected from 28° C. to 35° C. In a related embodiment the second temperature is about 1° C. to about 9° C. lower than the first temperature. In one embodiment the first temperature regime is a single temperature from 36° C. to 37° C. and the second temperature regime comprises at least one temperature shift from a first temperature of 36° C. to 37° C. to a second temperature of 28° C. to 35° C. In one embodiment the product-related impurity results from an imbalance in the ratio of long heavy chain to short heavy chain. In one embodiment the product-related impurity comprises an unpaired or mis-paired long heavy chain. In a related embodiment the product-related impurity comprises an unpaired or mis-paired long heavy chain and is selected from a homodimer, half antibody, protein aggregate, antibody fragment, combination of antibody fragments, and unpaired antibody fragments. In one embodiment the cell culture temperature regime may be further selected for modulation of the amount of expression, productivity, growth, yield, and/or other desired product quality attributes of the asymmetric multispecific antibody. In one embodiment the asymmetric multispecific antibody comprises a mutein. In one embodiment the asymmetric multispecific antibody is an asymmetric multispecific anti-PD-1 antibody comprising a single IL-21 mutein attached to one of the two antibody heavy chains of the anti-PD-1 antibody. In a related embodiment the long heavy chain comprises an anti-PD-1 antibody heavy chain having an attached IL-21 mutein.

The invention provides a method for modulating the product quality of a recombinant asymmetric multispecific antibody comprising a mutein expressed by a cell during cell culture comprising the steps: (a) establishing at least two cell cultures each inoculated with the same cell line expressing the asymmetric multispecific antibody; (b) culturing at least one cell culture at a first temperature regime that consists of a single temperature and at least one cell culture at a second temperature regime; (c) comparing at least one product-related impurity comprising an unpaired or mis-paired long heavy chain produced by a cell culture at each temperature regime; and (d) selecting the temperature regime that reduces the expression of at least one product-related impurity comprising an unpaired or mis-paired long heavy chain. In one embodiment the mutein is an IL-21 mutein. In a related embodiment the IL-21 mutein comprises amino acid substitutions at any two of positions 5, 9, 73, and 76 of SEQ ID NO: 1; wherein said amino acid substitutions are selected from: A, E, or Q at position 5. E or A at position 9. A or Q at position 73, and A, D, or E at position 76. In a related embodiment the IL-21 mutein comprises the amino acid sequence of any of SEQ ID NOs: 233-245. In one embodiment the asymmetric multispecific antibody comprises a mutein attached to the C-terminus of one of the two antibody heavy chains of an anti-PD-1 antibody. In a related embodiment the anti-PD-1 antibody comprising: two light chains, each comprising a LC CDR1, LC CDR2, and LC CDR3 comprising the amino acid sequence of SEQ ID NOs: 385, 386, and 387, respectively; and two heavy chains, each comprising a HC CDR1, LC CDR2, and LC CDR3 comprising the amino acid sequence of SEQ ID NOs: 382, 383, and 384, respectively. In a related embodiment the anti-PD-1 antibody comprises two light chains comprising the amino acid sequence of SEQ ID NO: 389 and two heavy chains comprising the amino acid sequence of SEQ ID NO: 388. In a related embodiment in the anti-PD-1 antibody comprises: two light chains, each comprising a LC CDR1, LC CDR2, and LC CDR3 comprising the amino acid sequence of SEQ ID NOs: 365, 366, and 367, respectively; and two heavy chains, each comprising a HC CDR1, HC CDR2, and HC CDR3 comprising the amino acid sequence of SEQ ID NOs:362, 363, and 364, respectively. In a related embodiment the anti-PD-1 antibody comprises two light chains comprising the amino acid sequence of SEQ ID NO: 369 and two heavy chains comprising the amino acid sequence of SEQ ID NO: 368. In a related embodiment the anti-PD-1 antibody comprises: (i) two light chains, each comprising the amino acid sequence of SEQ ID NO: 391; a heavy chain attached to an IL-21 mutein comprising the amino acid sequence of any one of SEQ ID NOs: 501-506;

and a heavy chain comprising the amino acid sequence of any one of SEQ ID NOs: 556-558; or (ii) two light chains, each comprising the amino acid sequence of SEQ ID NO: 371; a heavy chain attached to a single IL-21 mutein comprising an amino acid sequence of any one of SEQ ID NOs: 513-518; and a heavy chain comprising the amino acid sequence of any one of SEQ ID NOs: 559-561. In a related embodiment the anti-PD-1 antibody comprises: two light chains, each comprising the amino acid sequence of SEQ ID NO: 391; one heavy chain comprising the amino acid sequence of SEQ ID NO: 556; and one heavy chain attached to an IL-21 mutein comprising the amino acid sequence of SEQ ID NO: 501. In a related embodiment the anti-PD-1 antibody comprises: two light chains, each comprising the amino acid sequence of SEQ ID NO: 371; one heavy chain comprising the amino acid sequence of SEQ ID NO: 559; and one heavy chain attached to an IL-21 mutein comprising the amino acid sequence of SEQ ID NO: 513. In one embodiment the asymmetric multispecific antibody comprises an IL-21 mutein attached to the C-terminus of one of the two antibody heavy chains of an anti-PD-1 antibody. In one embodiment the long heavy chain comprises an anti-PD-1 antibody heavy chain having an attached IL-21 mutein.

The invention provides a method for modulating the product quality of a recombinant asymmetric multispecific antibody expressed by a cell during cell culture. The invention provides a method for modulating the product quality of a recombinant asymmetric multispecific antibody comprising a mutein expressed by a cell during cell culture. In particular, the invention provides a method for modulating the product quality of a conjugate of an IL-21 mutein linked to the C-terminus of one of the two antibody heavy chains of an anti-PD-1 antibody expressed by a cell during cell culture comprising the steps: (a) establishing a cell culture inoculated with a cell line expressing the antibody; (b) culturing the cells at 36±1° C. for the duration of the culture; and (c) harvesting the antibody; wherein amount of at least one product-related impurity resulting from an imbalance in the ratio of long heavy chain to short heavy chain in the harvested cell culture is decreased compared to the amount of the same product-related impurity in the harvest from a cell culture exposed to a temperature of 32° C. to 34° C. at some point during the culture.

The invention provides a method for selecting a cell line expressing an asymmetric multispecific antibody. The invention provides a method for selecting a cell line expressing an asymmetric multispecific antibody comprising a mutein comprising the steps: a) establishing at least one clonally derived cell line expressing the antibody; b) establishing at least two cell cultures from one or more of the clonally derived cell lines, wherein at least one cell culture from each cell line is cultured at a first temperature regime and at least one cell culture from each cell line is cultured at a second temperature regime; c) comparing the expression of at least one product-related impurity comprising an unpaired or mis-paired long heavy chain produced by the cells cultured at each temperature regime; and (d) selecting a cell line that resulted in reduced expression of at least one product-related impurity comprising an unpaired or mis-paired long heavy chain. In one embodiment the selected cell line is then cultured at the temperature regime that resulted in reduced expression of at least one product-related impurity comprising an unpaired or mis-paired long heavy chain. In one embodiment the first temperature regime is selected from about 36° C. to about 37° C. In one embodiment the second temperature regime comprises a temperature shift from a first temperature to a second temperature that is higher or lower than the first temperature. In a related embodiment the second temperature is selected from about 28° C. to about 35° C. In one embodiment the second temperature is about 1° C. to 9° C. lower than the first temperature. In one embodiment the first temperature regime is a single temperature from 36° C. to 37° C. and the second temperature regime comprises at least one temperature shift from a first temperature of 36° C. to 37° C. to a second temperature of 28° C. to 35° C. In one embodiment the product-related impurity comprising an unpaired or mis-paired long heavy chain results from an imbalance in the ratio of long heavy chain to short heavy chain. In a related embodiment the product-related impurity comprising an unpaired or mis-paired long heavy chain results from an increase in the ratio of the long heavy chain to the short heavy chain. In one embodiment the product-related impurity comprising an unpaired or mis-paired long heavy chain and is selected from a homodimer, half antibody, protein aggregate, antibody fragment, combination of antibody fragments, and unpaired antibody fragments. In one embodiment the cell culture temperature regime may be further selected for a temperature regime that also modulates the amount of expression, productivity, growth, and/or other desired product quality attributes of the asymmetric multispecific antibody. In one embodiment the asymmetric multispecific antibody is an asymmetric multispecific anti-PD-1 antibody comprising a single IL-21 mutein attached to one of the two antibody heavy chains of the anti-PD-1 antibody. In one embodiment the long heavy chain comprises an anti-PD-1 antibody heavy chain having an attached IL-21 mutein.

The invention provides a method for modulating production of at least one recombinant asymmetric multispecific antibody product-related impurity comprising an antibody heavy chain. The invention provides a method for modulating production of at least one recombinant asymmetric multispecific antibody product-related impurity comprising a long heavy chain. In particular the invention provides a method for modulating production of at least one recombinant asymmetric multispecific antibody product-related impurity comprising an antibody heavy chain having an attached IL-21 mutein during cell culture through cell culture temperature comprising the steps: selecting the temperature regime that reduces the expression of at least one product-related impurity comprising an unpaired or mis-paired long heavy chain comprising a long heavy chain; (a) culturing the cell line at the selected temperature regime; and (b) harvesting the recombinant asymmetric multispecific antibody. In one embodiment the asymmetric multispecific antibody is an asymmetric multispecific anti-PD-1 antibody comprising a single IL-21 mutein attached to one of the two antibody heavy chains of the anti-PD-1 antibody. In one embodiment the long heavy chain comprises an anti-PD-1 antibody heavy chain having an attached IL-21 mutein.

The invention provides a method for producing an isolated, purified, recombinant asymmetric multispecific antibody, the method comprising the steps: a) isolating at least one single stably transformed cell expressing the asymmetric multispecific antibody and establishing a clonally derived culture; b) establishing at least two cell cultures from one or more of the clonally derived cultures; c) culturing at least one cell culture from each clonally derived culture at a first temperature regime comprising a single temperature and culturing at least one cell culture from each clonally derived culture at a second temperature regime; d) comparing the amount of at least one product-related impurity comprising an unpaired or mis-paired long heavy chain produced by the cells cultured at each temperature regime; e) selecting the cell culture that modulated production of the product related impurity; f) establishing a cell line expressing the asymmetric multispecific antibody from the selected cell culture; g) inoculating a bioreactor with the cell line expressing the asymmetric multispecific antibody; h) culturing the cells to express the asymmetric multispecific antibody at the temperature regime that modulated the production of the product-related impurity; i) harvesting the recombinant asymmetric multispecific antibody from the cell culture; j) processing the recombinant multispecific antibody through one or more chromatography unit operations; and k) obtaining an isolated, purified, recombinant asymmetric multispecific antibody. In one embodiment is provided an isolated, purified, recombinant multispecific antibody according to the method above. In one embodiment is provided a pharmaceutical composition comprising the isolated, purified, recombinant multispecific antibody according to the method above. In one embodiment the asymmetric multispecific antibody is an asymmetric multispecific anti-PD-1 antibody comprising a single IL-21 mutein attached to one of the two antibody heavy chains of the anti-PD-1 antibody. In one embodiment the long heavy chain comprises an anti-PD-1 antibody heavy chain having an attached IL-21 mutein.

The invention provides a method for controlling the growth of cells in a nanofluidic chamber of a nanofluidic chip comprising the steps of: (a) isolating a single cell in to a nanofluidic chamber of a nanofluidic chip, wherein said cell comprises an expression construct capable of expressing a recombinant protein; (b) culturing the cell at a first temperature; (c) at a predetermined point, culturing the cells at a second temperature; and (d) exporting the cells out of the nanofluidic chamber and into a culture vessel.

The invention also provides a method for minimizing cross contamination during export of cells grown in a nanofluidic chamber of a nanofluidic chip comprising the steps of: (a) isolating a single cell in to a nanofluidic chamber of a nanofluidic chip, wherein said cell comprises an expression construct capable of expressing a recombinant protein; (b) culturing the cell at a first temperature; (c) at a predetermined point, culturing the cells at a second temperature; and (d) exporting the cells out of the nanofluidic chamber and into a culture vessel; wherein the number of cells per chamber at export is lower compared to a single cell cultured under similar conditions a constant temperature for the duration of the culture.

The invention further provides a method to improve clone selection for cells grown in a nanofluidic chamber of a nanofluidic chip comprising the steps of: (a) isolating a single cell in to a nanofluidic chamber of a nanofluidic chip, wherein said cell comprises an expression construct capable of expressing a recombinant protein; (b) culturing the cell at a first temperature; (c) lowering the temperature of the culture to a second temperature no earlier than day 3 of the culture; and (d) exporting the cells out of the nanofluidic chamber at least four days after the temperature shift.

In one embodiment the number of cells per chamber at export is lower compared to a single cell cultured under similar conditions at a constant temperature for the duration of the culture. In one embodiment the first temperature is selected from 35° C. to 37° C. In one embodiment the second temperature is selected from 28° C. to 34° C. In one embodiment the second temperature is about 1° C. to about 9° C. lower than the first temperature. In one embodiment the first temperature is 36° C. and the second temperature is 32° C. to 32.5° C. In one embodiment the predetermined point is on day 3 to day 5 of the culture. In one embodiment export is on day 6 to day 8 of the culture. In one embodiment the nanofluidic chip comprises 1758 chambers, 3,500 chambers, 11,000 chambers, 14,000 chamber, or 20,000 chambers. In one embodiment the culture vessel is a multi-well plate. In one embodiment the protein secretion profiles before and after the temperature shift are compared.

### BRIEF DESCRIPTION OF THE FIGURES

FIG. 1 (A) Shows an increase in the % long heavy chain (% LHC) when cells expressing an IL-2-anti-PD-1 conjugate were subjected to perfusion culture (P) with a temperature shift to 32.5° C. (open bars), compared to fed batch culture (FB) at a constant 36° C. (striped bars). (B) and (C) Show the results of the production of an IL-2-anti-PD-1 conjugate expressed by cells in a perfusion culture (P) with a temperature shift from 36° C. to 32.5° C. subjected to purification by Protein A affinity chromatography (left open bar) and cation exchange chromatography (right open bar) compared to the production of an IL-2-anti-PD-1 conjugate expressed by cells in a constant temperature (36° C.) fed batch culture (FB) subjected to Protein A affinity chromatography only (striped bar). (B) % Pre-Peaks by nrCE. (C) % low molecular weight impurities (LMW) by SEC. (D) Shows impurities characterization by nr CE-SDS from the CEX pool. Excess long heavy chain could not be separated by CEX.

FIG. 2 Shows that there was better correlation between the fed batch samples and the perfusion samples when they were screened at the same temperature, comparing the Molar Ratio of the long heavy chain (LHC) to the short heavy chain (SHC) as determined by rCE. (A) shows fed batch culture samples grown at a constant 36° C. compared to perfusion samples grown with a temperature shift (TS) from 36° C. to 32.5° C. Fed Batch (x, constant 36° C.) vs. Perfusion (y, 36° C. to 32.5° C.). (B) shows fed batch culture samples compared to perfusion samples that were both grown with a temperature shift (TS) from 36° C. to 32.5° C. Fed Batch on the x axis vs. Perfusion on the y axis. The data points for both culture methods grown at a constant 36° C. were limited and not included in the comparison.

FIG. 3 Shows that there was better correlation between the fed batch samples and the perfusion samples if they were screened at the same temperature, comparing the percent low molecular weight (% LMW) determined by SEC. (A) shows fed batch culture samples grown at a constant 36° C. compared to perfusion samples grown with a temperature shift (TS) from 36° C. to 32.5° C. Fed Batch on the x axis vs. Perfusion on the axis. (B) shows fed batch culture samples compared to perfusion samples, both grown with a temperature shift (TS) from 36° C. to 32.5° C. Fed Batch on the x axis vs. Perfusion on the y axis. The data points for both culture methods grown at a constant 36° C. were limited and not included in the comparison.

FIG. 4 Shows the results of the production of an IL-2-anti-PD-1 conjugate expressed by cells in a perfusion culture at a constant temperature of 36° C. compared to a perfusion culture with a temperature shift from 36° C. to 32.5° C. (A) shows the Molar Ratio of the long heavy chain (LHC) to the short heavy chain (SHC) as determined by rCE (Constant 36° C., black bars. Temperature shift from 36° C. to 32.5° C., gray bars). (B) shows the % Pre-Peak 6 (impurities formed by SHC) as determined by nrCE, purification by Protein A affinity chromatography (black bars) and cation exchange chromatography (gray bars). (C) shows % Pre-Peak 7 (impurities formed by subjected by LHC) as determined by nrCE, purification by Protein A affinity chromatography (black bars) and cation exchange chromatography (gray bars).

FIG. 5 Shows the results of Bispecific A clone 1 production by fed batch culture when temperature is held at constant 36° C. (black bar) and with temperature shift from 36° C. to 32.5° C. (gray bar). (A) Molar ratio of long heavy chain (LHC) to short heavy chain (SHC) as determined by rCE-SDS. (B) % HMW by SEC. (C) % LMW by SEC (impurities formed by LHC).

FIG. 6 Shows the results of Bispecific A clones 2 and 3 production by mock perfusion culture when temperature is held at constant 36° C. (black bar) and with temperature shift from 36° C. to 32.5° C. (gray bar). (A) Molar ratio of long heavy chain (LHC) to short heavy chain (SHC) as determined by rCE-SDS. (B) % LMW as determined by SEC. (C) Transcript ratio of LHC to SHC as determined by ddPCR.

FIG. 7 Shows the results of Bispecific B clones 1, 2, and 3 production by fed batch culture when temperature is held at constant 36° C. (black bar) and with temperature shift from 36° C. to 32.5° C. (gray bar). (A) Molar ratio of the long heavy chain (LHC) to the short heavy chain (SHC) as determined by rCE-SDS. (B) % HMW2 as determined by SEC. (C) % LMW1 as determined by SEC. (D) Transcription ratio of LHC to SHC as determined by ddPCR.

FIG. 8 Shows the results of Bispecific B clones 2, and 3 production by mock perfusion comparing culture at a constant temperature of 36° C. (black bar) with a temperature shift from 36° C. to 32.5° C. (gray bar). (A) Molar ratio of the long heavy chain (LHC) to the short heavy chain (SHC) as determined by rCE-SDS. (B) % HMW2 as determined by SEC. (C) % LMW1 as determined by SEC.

FIG. 9 Shows that temperature shift implemented during on chip culture results in growth inhibition and allows for long-term culture. (A) Representative brightfield chamber images collected throughout experiment duration on Berkeley Lights Bacon platform, where single cells expressing monoclonal antibody were loaded on a chip into individual chambers and the chip was cultured for 6 days at 36° C. The cell counts generated by the instrument software are displayed below each timepoint. Cell population on day 6 occupied majority of the chamber content and expanded close to the neck area of the chamber, preventing reliable secretion assessment through Spotlight™ Human Fc Assay and creating high risk of cross contamination during potential export procedure. (B) Representative brightfield chamber images collected throughout experiment duration on Berkeley Lights Bacon platform, where single cells expressing monoclonal antibody were loaded on a chip into individual chambers and the chip was cultured for 3 days at 36° C. and temperature was subsequently reduced to 32° C. for the remaining experiment duration. The cell counts generated by the instrument software are displayed below each timepoint. Temperature shift implemented from Day 4 resulted in growth inhibition and prevented cell expanding above the maximum recommended height of the chamber. (C) Average doubling time (top panel) and average number of cells per chamber (bottom panel) measured on each day throughout experiment duration. Implementing temperature shift from Day 4 resulted in growth inhibition manifested by increased average doubling time and decreased average cell counts when compared to the control condition.

FIG. 10 Shows that implementing temperature shift during CLD workflow on the Berkeley Light Beacon platform alters monoclonal antibody production profiles. (A) Schematic representation of standard Cell Line Development cloning workflow on the Beacon platform (left panel) and Cell Line Development cloning workflow with temperature shift (right panel) implemented in B and C. (B) Normalized secretion score data corresponding to Spotlight™ Human Fc Assay conducted before temperature shift on Day 4 (left panel) and after temperature shift on Day 6 (right panel). Cell secretion profiles measured after temperature shift showed more diverse distribution allowing better distinction of highly producing clones form poorly secreting cell lines. (C) Brightfield and Spotlight™ Human Fc Assay fluorescent images collected throughout experiment duration on Berkeley Lights Bacon platform corresponding to two clonally derived cell lines expressing monoclonal antibody where CLD workflow with temperature shift was implemented. The cell counts generated by the instrument software and normalized Secretion score are displayed below each timepoint. Incubation in decreased temperature conditions resulted in growth arrest for both clones. While both cell lines demonstrated comparable secretion profiles on Day 4 of the workflow. Clone 1 but not Clone 2 displayed increased secretion levels in response to temperature shift.

FIG. 11 Shows that implementing temperature shift during CLD workflow on the Berkeley Light Beacon platform achieves growth inhibition and alters recombinant protein production profiles for cell lines expressing IgG4 monoclonal antibodies and multispecific antibody formats. (A)(D) Average doubling time (top panel) and average number of cells per chamber (bottom panel) measured on each day throughout experiment duration for clonally derived cell lines expressing IgG4 monoclonal antibody and multispecific antibody, respectively. Temperature settings for each timepoint of the experiment are outlined on the top of the graph. Implementing temperature shift from Day 4 or Day 3, respectively, resulted in growth inhibition measured by increased doubling time and decreased cell counts for both IgG4 and multispecific antibody expressing cell lines. (B)(E) Representative brightfield chamber images collected throughout experiment duration on Berkeley Lights Bacon platform of clonally derived cell lines expressing monoclonal antibody or multispecific antibody, respectively. The cell counts generated by the instrument software are displayed below each timepoint. (C)(F) Spotlight™ Human Fc Assay score data corresponding to Day 4 and Day 8 or Day 7 timepoints, respectively. Secretion profiles achieved after prolonged incubation in decreased temperature showed more diverse distribution allowing better distinction of highly producing clones form poorly secreting cell lines.

FIG. 12 Shows that export procedure conducted at 32° C. temperature results in similar clone recovery when compared to export executed at 36° C. per standard CLD workflow. (A)(B) Top panel: schematic representation of standard Cell Line Development cloning workflow on the Beacon platform and Cell Line Development cloning workflow with temperature shift implemented during export process, respectively. Bottom panel: representative images of 96 well export plates acquired 18 days after export procedure showing comparable clone recovery efficiency regardless of export temperature implemented at the single cell cloning stage.

### DETAILED DESCRIPTION OF THE INVENTION

Monoclonal antibodies are symmetrical, having two identical polypeptide chains, each pair having one “light” chain (LC) and one “heavy” chain (HC), and are specific for one target. Multispecific antibodies that bind, neutralize, and/or interact with more than one target are being engineered in an ever-increasing variety of compositions and configurations and in an increasing variety of formats to meet ever more challenging therapeutic indications that offer alternatives and improvements over monoclonal antibodies. Many multispecific antibodies share characteristics with monoclonal antibodies, such as having one light and one heavy chain, however, unlike monoclonal antibodies, the heavy chains of multispecific antibodies can be asymmetric.

“Asymmetric” multispecific antibodies comprise heavy chains that differ in length. A heavy chain typically comprises a fragment antigen binding (FAB) region comprising a variable domain (Vh) and a constant domain (C_{H}1), a fragment crystallizable (Fc) region that comprises two constant domains (C_{H}2 and/or C_{H}3), and a hinge region connecting the Fab and Fc regions. Asymmetric multispecific antibody heavy chains may comprise some or all of these components. In addition, the heavy chain may also comprise one or more additional components, attached directly and/or indirectly to any point in a variable domain and/or a constant domain of the heavy chain. The length of the heavy chain is determined based on the combined size of the amino acid sequences of all the components that make up the heavy chain, including any variable domain(s), constant domain(s), hinge regions, attached proteins and/or peptides and any linkers and/or other attachment mechanisms. The heavy chain having the longest combined amino acid sequence is referred to as the “long heavy chain”. The heavy chain with the shorter combined amino acid sequence is referred to as the “short heavy chain”. Asymmetric multispecific antibodies have at least one long heavy chain and at least one short heavy chain. The asymmetric multispecific antibodies bind, neutralize, and/or interact specifically with the at least two different antigens or targets, and/or different epitopes on the same antigen or target.

In one embodiment the long heavy chain of an asymmetric antibody comprises an attached mutein. In one embodiment the asymmetric multispecific antibody is an anti-PD1 antibody comprising a long heavy chain and a short heavy chain. The long heavy chain comprises a single IL-21 mutein attached to one of the two antibody heavy chains of the anti-PD-1 antibody, the short heavy chain comprising the other anti-PD-1 antibody heavy chain. In one embodiment the long heavy chain comprises an IL-21 mutein attached to the Fc of the anti-PD-1 antibody. In one embodiment the long heavy chain comprises an IL-21 mutein directly attached to the Fc of the anti-PD-1 antibody without a linker. In one embodiment the long heavy chain comprises an IL-21 mutein directly attached to the C-terminus of one of the two antibody heavy chains of the anti-PD-1 antibody with or without a linker.

Producing and purifying asymmetric multispecific antibodies brings its own manufacturing challenges. The engineered origin and asymmetry of these antibodies makes them susceptible to formation of product-related impurities throughout the manufacturing process, especially during cell culture operations. The type and/or quantity of the impurity may impact the expression, purification, yield, activity, and/or product quality of these asymmetric multispecific antibodies. “Product-related impurity” refers to variants that may share one or more structural components with the desired product, such as an asymmetrical multispecific antibody. One example of a structural component is a long heavy chain of an asymmetric multispecific antibody. When such impurities are expressed during cell culture, they must be removed in downstream purification operations. The challenge to removing these impurities is increased depending on how similar the impurities are to the protein of interest, such as in composition, size, hydrophobicity, isoelectric point (pI), surface charge, and the like.

Balanced expression of the unique components that make up recombinant asymmetric multispecific antibodies is a challenge when expressing these proteins in culture. An imbalance in the expression of one or more components can lead to the formation of product-related impurities, such as unpaired and/or mis-paired protein variants. The degree of success in removing the product-related impurities can depend on the properties of the impurities. In addition, removal operations will result in some degree of product loss. As described herein, product-related impurities comprising an anti-PD-1 long heavy chain were found to be unexpectedly difficult to remove during purification operations and negatively impacted product quality, yield, and the robustness of the manufacturing process as a whole.

The molar ratio of long heavy chain to short heavy chain of asymmetrical multispecific antibodies that were produced during cell culture was found to be influenced by the culture temperature. As described in more detail herein and in the Examples, it was discovered that when clonal cells expressing asymmetric multispecific antibodies were subjected to a low temperature set point during the cell culture operation, as a result of a temperature shift (e.g., 36° C. to 34° C. or 32.5° C.). The molar ratio of long heavy chain to short heavy chain expressed by the clonal cells unexpectedly increased at the lower culture temperatures. In this instance, clonal cells expressing asymmetric multispecific antibodies that had been grown and selected using a typical cell line development processes that maintained a stationary, physiological temperature (e.g., 36° C.) during single cell cloning, scale up, and selection, as is typically done for clonal cells expressing other types of proteins such as monoclonal antibodies and symmetric multispecific antibodies. In one embodiment, the product-related impurity results from an imbalance in heavy chain expression. In one embodiment, the product-related impurity results from an imbalance in the ratio of long heavy chain to short heavy chain. In one embodiment, the product-related impurity results from an increase in the ratio of the long heavy chain to short heavy chain. No impact due to the pH of the cell culture was observed.

Temperature shifts are commonly used during the cell culture operations to influence cell growth and recombinant protein production of symmetric proteins. However, for cells expressing asymmetric multispecific antibodies such a temperature shift during cell culture may unexpectedly alter the ratio of the long heavy chain and short heavy chain that are expressed during the culture. The increase in the ratio of long heavy chain to short heavy chain was found to influence the formation and the type of product-related impurities. This altered ratio of the long and short heavy chains can impact the purification, activity, product quality, as well as the robustness of the manufacturing process as a whole.

It was found that the product quality of asymmetric multispecific antibodies could be modulated through the cell culture temperature. In particular, the invention provides a method for determining the temperature conditions which maintain a more balanced expression of long heavy chain to short heavy, and thereby reducing the formation of product-related impurities having unpaired and/or mis-paired long heavy chains. In one embodiment, the cell culture temperature regime may be further selected such that in addition to modulating heavy chain-related product-related impurities, the temperature also modulates expression, productivity, growth, and/or other desired product quality attributes of the asymmetric multispecific antibody.

As described herein, the impact of the unexpected temperature-induced imbalance of the expression of the long heavy chain of an asymmetric multispecific antibody was problematic for the manufacture of drug products comprising such proteins. Symmetric proteins, such as monoclonal antibodies, balanced expression of heavy chains may not be as critical since the chains are identical. However, for asymmetric multispecific antibodies having at least one long heavy chain and at least one short heavy chain, balancing expression of the heavy chain was found to be critical component of a robust manufacturing operation and the production of drug product having the desired yield and product quality attributes. An imbalance in the production of the long and short heavy chains increased the opportunity for the formation of product-related impurities resulting from unpaired or mis-paired long heavy chains. In one embodiment, the product-related impurity results from an imbalance in heavy chain expression. In one embodiment, the product-related impurity comprises an unpaired or mis-paired heavy chain. In one embodiment, the product-related impurity comprises an unpaired or mis-paired long heavy chain.

Product-related impurities include unpaired and/or mis-paired heavy chains. Exemplary product-related impurities include unpaired long heavy chains and/or mis-paired long heavy chains. Product-related impurities may be in the form of homodimers, half antibodies, aggregates, unpaired components, antibody fragments and/or various combinations of antibody fragments, and the like that comprise a long heavy chain. “Half antibodies” refer to a product-related impurity that can form, for example, due to incomplete assembly or disruption of the interaction between the two heavy chain polypeptides. Half antibodies comprise a single light chain polypeptide and a single heavy chain polypeptide. “Homodimers” refer to a product-related impurity that can form, for example, when heavy and light chains having specificity for the same target recombine with each other instead of pairing with heavy and light chains that have specificity to a different target to form a desired asymmetric multispecific heterodimer. This typically occurs during expression in the host cell. Product-related impurities in the form of homodimers, half antibodies, aggregates, antibody fragments and various combinations of antibody fragments, unpaired components, and the like include those having at least one long heavy chain.

In one embodiment, the product-related impurity comprises an unpaired long heavy chain. In one embodiment, the product-related impurity comprises a mis-paired long heavy chain. In one embodiment the product-related impurity is selected from a homodimer, half antibody, protein aggregate, antibody fragment, combination of antibody fragments, and unpaired components having an unpaired or mis-paired heavy chain. In one embodiment the product-related impurity is selected from a homodimer, half antibody, protein aggregate, antibody fragment, combination of antibody fragments, or unpaired antibody fragments having an unpaired heavy chain. In one embodiment the product-related impurity is selected from a homodimer, half antibody, protein aggregate, antibody fragment, or combination of antibody fragments having a mis-paired heavy chain. In one embodiment the product-related impurity is selected from a homodimer, half antibody, protein aggregate, antibody fragment, combination of antibody fragments, and unpaired components having an unpaired or mis-paired long heavy chain. In one embodiment the product-related impurity comprises a homodimer comprising a long heavy chain. In one embodiment the product-related impurity comprises a half-antibody comprising a long heavy chain. In one embodiment the product-related impurity comprises an aggregate comprising a long heavy chain. In one embodiment the product-related impurity comprises an antibody fragment comprising a long heavy chain. In one embodiment the product-related impurity comprises an unpaired antibody fragment comprising a long heavy chain.

The invention provides a method for selecting a cell culture temperature regime that modulates the amount of at least one asymmetric multispecific antibody product-related impurity comprising the steps: a) establishing at least two cell cultures each inoculated with the same cell line expressing the asymmetric multispecific antibody; b) culturing at least one cell culture at a first temperature regime that consists of a single temperature and at least one cell culture at a second temperature regime; c) comparing the amount of at least one product-related impurity in the cell culture at each temperature regime; and d) selecting the temperature regime that reduces the amount of the product-related impurity in the cell culture.

The invention provides a method for modulating the product quality of a recombinant asymmetric multispecific antibody expressed by a cell during cell culture comprising the steps: a) selecting the temperature regime that reduces the expression of at least one product-related impurity resulting from an imbalance in heavy chain expression, the impurity comprising an unpaired or mis-paired long heavy chain; culturing the cell line at the selected temperature regime; and harvesting the recombinant asymmetric multispecific antibody.

In one embodiment selecting the temperature regime comprises the steps of a) establishing at least two cell cultures each inoculated with the same cell line expressing the asymmetric multispecific antibody; b) culturing at least one cell culture at a first temperature regime that consists of a single temperature and at least one cell culture at a second temperature regime; c) comparing at least one product-related impurity in the cell culture at each temperature regime; and d) selecting the temperature regime that reduces the expression of at least one product-related impurity comprising an unpaired or mis-paired long heavy chain.

The invention provides a method for modulating the product quality of a recombinant asymmetric multispecific antibody expressed by a cell during cell culture comprising the steps: a) selecting the temperature regime that reduces the expression of at least one product-related impurity comprising an unpaired or mis-paired long heavy chain; b) culturing the cell line at the selected temperature regime; and c) harvesting the recombinant asymmetric multispecific antibody.

The invention provides a method for modulating production of at least one recombinant asymmetric multispecific antibody product-related impurity comprising a long heavy chain during cell culture through cell culture temperature comprising the steps a) selecting the temperature regime that reduces the expression of at least one product-related impurity comprising an unpaired or mis-paired long heavy chain comprising a long heavy chain; b) culturing the cell line at the selected temperature regime; and c) harvesting the recombinant asymmetric multispecific antibody.

The development of biotherapeutics relies on manipulating mammalian cells to secrete desired proteins. Successful development of a biotherapeutic is a lengthy, multiple step process that includes development of a clonal cell line expressing the biotherapeutic. Typical mammalian cell line development processes are resource intensive and require lengthy timelines due in part to the slow recovery of cells during selection, single cell cloning, and the need to perform multiple screening assays to identify suitable production hosts. Clone selection is one of the most labor-intensive steps during cell line development because of the need to screen large numbers of candidates in various protein production studies. The desired outcome from this process is the identification and isolation of a mammalian clonal cell line that expresses a therapeutic protein having desired product quality attributes while also conforming to desired manufacturing processes and operations with minimal impact.

Clonal cell line selection includes attributes desired in the expressed recombinant protein, such as titer, product quality, and/or growth characteristics, which can include a more balanced molar ratio of the heavy chains. When clonal cell line candidates that were selected under a single, constant temperature condition were grown in a bioreactor under conditions that incorporated at least one change or shift in temperature, the titer of the expressed recombinant protein and viable cell density were minimally impacted, but the molar ratio of long heavy chain to short heavy chain increased, and as a result, the quantity and type of product-related impurities that were expressed also changed which impacted the yield, product quality attributes of the desired protein, the downstream purification of the protein, and the robustness of the manufacturing process as a whole.

The imbalance in the ratio of the asymmetric heavy chains impacted the types of product-related impurities that were formed. It was found that excess production of the long heavy chain resulted in an increase in protein-related impurities comprising the long heavy chain, such as homodimers and monomers containing the long heavy chain. The unbalanced expression of the heavy chains and the need to remove the impurities formed due to the imbalance in expression, which was unexpectedly found to be difficult with certain long heavy chain containing impurities, impacted the yield and product quality attributes of asymmetric multispecific antibodies. As such, it is desirable to maintain a more balanced expression of the asymmetric heavy chain to maintain the desired and/or required product quality attributes, as well as reducing unnecessary time, resources, product loss and decrease in the robustness of the manufacturing process due to the excessive effort to recover the product from the undesired long heavy chain impurity.

Delaying production of a desired drug substance to switch cell lines or rescreen clones to identify new cell lines that are capable of reliably expressing the recombinant asymmetric multispecific antibody drug substance that conforms to desired product qualities and attributes, and is amenable to established facility practices and/or protocols, such as temperature shifts, is costly in terms of time, resources, manpower, and does not support an efficient and effective manufacturing process. Therefor use of temperature as a factor during selection of clonal cell lines expressing asymmetric multispecific antibodies would be beneficial for obtaining production cell lines that express the desired protein with the desired qualities and thereby reducing and/or eliminating the production of product-related impurities that impact the production of the desired protein.

It was found that temperature could be used as a lever during cell line development to select desirable cell line candidates expressing recombinant asymmetric multispecific antibodies that meet product quality as well as manufacturability requirements. Cell culture temperature has the potential to influence the product quality of asymmetric proteins, such as by altering the molar ratio of long heavy chain to short heavy chain which influenced the management of product-related impurities. It would be beneficial for a cell line development process to incorporate desired set point temperatures during the screening and selection processes that meet the requirements of not only the drug product attributes, but also desired facility practices and/or protocols. Clone screening using temperature as a lever is also beneficial as product quality attribute response to temperature appears to be molecule dependent and/or cell line dependent. Modulating product quality using temperature during screening and selection of clonal cell lines reduced selection of clonal cell lines that were more susceptible to expression of product-related impurities, improved product quality, and were more amenable to desired process practices.

The invention provides a method for selecting a cell line expressing an asymmetric multispecific antibody comprising the steps: establishing at least one clonally derived cell line expressing the asymmetric multispecific antibody; establishing at least two cell cultures from one or more of the clonally derived cell lines, wherein at least one cell culture from each cell line is cultured at a first temperature regime and at least one cell culture from each cell line is cultured at a second temperature regime; comparing the expression of at least one product-related impurity produced by the cells cultured at each temperature regime; and selecting a cell line that resulted in reduced expression of at least one product-related impurity. In one embodiment the selected cell line is then cultured at the temperature regime that resulted in reduced expression of at least one product-related impurity.

The invention provides a method for establishing a cell line from a clonally derived culture expressing an asymmetric multispecific antibody comprising the steps: transforming cells with at least one gene encoding an asymmetric multispecific antibody; isolating at least one single stably transformed cell expressing the asymmetric multispecific antibody and establishing a clonally derived culture; establishing at least two cell cultures from one or more clonally derived culture; culturing at least one cell culture from each clonally derived culture at a first temperature regime and culturing at least one cell culture from each clonally derived culture is cultured at a second temperature regime; comparing the molar ratio of long heavy chain to short heavy chain produced by the cells at each temperature regime; selecting at least one clonally derived culture that produced a lower molar ratio of long heavy chain to short heavy chain; and establishing a cell line from the clonally derived culture.

The invention provides a method for producing an isolated, purified, recombinant asymmetric multispecific antibody, the method comprising the steps: a) isolating at least one single stably transformed cell expressing the asymmetric multispecific antibody and establishing a clonally derived culture; b) establishing at least two cell cultures from one or more of the clonally derived cultures; c) culturing at least one cell culture from each clonally derived culture at a first temperature regime comprising a single temperature and culturing at least one cell culture from each clonally derived culture at a second temperature regime; d) comparing at least one related impurity comprising an unpaired or mis-paired long heavy chain produced by the cells cultured at each temperature regime; e) selecting the cell culture that modulated production of the product related impurity; f) establishing a cell line expressing the asymmetric multispecific antibody from the selected cell culture; g) inoculating a bioreactor with the cell line expressing the asymmetric multispecific antibody; h) culturing the cells to express the asymmetric multispecific antibody at the temperature that modulated the production of the product-related impurity; i) harvesting the recombinant asymmetric multispecific antibody from the cell culture; j) processing the recombinant asymmetric multispecific antibody through one or more chromatography unit operations; and k) obtaining an isolated, purified, recombinant asymmetric multispecific antibody.

The invention provides an isolated, purified, recombinant asymmetric multispecific antibodies according to the method described herein.

Cell lines suitable for production of recombinant asymmetric multispecific antibodies need to be stable with respect to product quality and productivity, without the expression or production, or increased expression or production, of undesirable product-related impurities. These cell lines must be capable of producing the desired asymmetric multispecific antibodies, with the same quality and attributes every time, over time.

Creation of stable cell lines begins with the transformation of a host cell that is suitable or desirable for recombinant production of the protein of interest. Typically, such cells are created, modified, and/or developed for production of recombinant proteins.

The cells may be derived from a multi-cellular animal. A commonly used animal cell line for biopharmaceutical production is a mammalian cell line. A wide variety of mammalian cell lines suitable for use are available from the American Type Culture Collection (Manassas, Va.) and commercial vendors. Commonly used cell lines include those from Chinese hamster ovary (CHO) cells, human embryonic kidney (HEK) cells, murine myeloma (NS0, Sp2/0) cells, baby hamster kidney (BHK) cells, human embryonic kidney (293) cells, fibrosarcoma (HT-1080) cells, human embryonic retinal (PER.C6) cells, hybrid kidney and B cells (HKB-11), CEVEC's amniocyte production (CAP) cells, human liver (HuH-7) cell, and any other cells that are used or suitable for use in clinical and/or commercial manufacturing.

The most commonly used cell lines are from Chinese hamster ovary (CHO) cells. CHO cells are widely used to produce complex recombinant proteins. The dihydrofolate reductase (DHFR)-deficient mutant cell lines (Urlaub et al. (1980), Proc Natl Acad Sci USA 77: 4216-4220), DXB11 and DG-44, are desirable CHO host cell lines because the efficient DHFR selectable and amplifiable gene expression system allows high level recombinant protein expression in these cells (Kaufman R. J. (1990). Meth Enzymol 185:537-566). The glutamine synthetase (GS)-knockout CHOK1SV cell lines, making use of glutamine synthetase (GS)-based methionine sulfoximine (MSX) selection are also widely used. Also included are CHOK1 cells (ATCC CCL61). In addition, these cells are easy to manipulate as adherent or suspension cultures and exhibit relatively good genetic stability. CHO cells and proteins recombinantly expressed by them have been extensively characterized and have been approved for use in clinical and commercial manufacturing by regulatory agencies.

The cells are transformed with an expression system(s), such as vectors comprising the gene(s) encoding the protein(s) of interest. Expression systems and constructs in the form of plasmids, expression vectors, transcription or expression cassettes that comprise at least one nucleic acid molecule encoding a protein of interest are also provided herein, as well host cells comprising such expression systems or constructs. As used herein, “vector” means any molecule or entity (e.g., nucleic acid, plasmid, bacteriophage, transposon, cosmid, chromosome, virus, virus capsid, virion, naked DNA, complexed DNA and the like) suitable for use to transfer and/or transport protein encoding information into a host cell and/or to a specific location and/or compartment within a host cell. Vectors can include viral and non-viral vectors, and non-episomal mammalian vectors. Vectors are often referred to as expression vectors, for example, recombinant expression vectors, or cloning vectors. The vector may be introduced into a host cell to allow replication of the vector itself and thereby amplify the copies of the polynucleotide contained therein. The cloning vectors may contain sequence components and generally include, without limitation, an origin of replication, promoter sequences, transcription initiation sequences, enhancer sequences, and selectable markers. These elements may be selected as appropriate by a person of ordinary skill in the art.

Transformation refers to a change in a cell's genetic characteristics. A cell has been transformed when it has been modified to contain new DNA or RNA. For example, a cell is transformed where it is genetically modified from its native state by introducing new genetic material via transfection, transduction, or other techniques. Transduction refers to the process whereby foreign DNA is introduced into a cell via viral vector. Transfection refers to the uptake of foreign or exogenous DNA by a cell. One or more vectors may be inserted into a suitable cell for amplification and/or polypeptide expression. The transformation of an expression vector into a selected cell may be accomplished by well-known methods including transfection, infection, calcium phosphate co-precipitation, electroporation, nucleofection, microinjection, DEAE-dextran mediated transfection, cationic lipids mediated delivery, liposome mediated transfection, microprojectile bombardment, receptor-mediated gene delivery, delivery mediated by polylysine, histone, chitosan, and peptides. The method selected will in part be a function of the type of host cell used. These methods and other suitable methods are well known to the skilled artisan.

Following transfection or transduction, the transforming DNA can recombine with that of the cell by physically integrating into a chromosome of the cell or can be maintained transiently as an episomal element without being replicated or can replicate independently as a plasmid. A cell is considered to have been “stably transformed” when the transforming DNA is replicated with the division of the cell. Following transfection, the viability of the cells typically decreases, and the unamplified pools are typically passaged multiple times to allow recovery. The pools are maintained as constant temperature cultures, typically a physiological temperature, such as 35° C.-37° C. typically 36° C.

The cells may be subjected to selective pressure to select those cells that have internalized the expression system. Selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include glutamine synthetase (GS)/methionine sulfoximine (MSX) system, dihydrofolate reductase (DHFR), and promoterless thymidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes a protein of interest. As a result, increased quantities of a polypeptide of interest are synthesized from the amplified DNA.

Two common systems used with CHO cells are DHFR-deficient cells (examples include CHO-DXB11 and CHO-DG44) and CHO cells lacking glutamine synthetase (GS) such as CHOK1SV or other GS-knock out cells. DHFR-deficient cells use of selection media lacking thymidine and hypoxanthine results in the survival of cells with sufficient exogenously integrated DHFR gene copies into the cells. Likewise, for cells lacking GS, use of selection media supplemented with L-glutamic acid instead of L-glutamine results in survival of cells having sufficient GS integrated into the cell genome.

Cell lines for commercial scale production of recombinant protein drug substance are derived from a single cell progenitor. These clonally derived cultures must be individually propagated and assessed for growth, productivity, and secreted protein product quality. Stable clonal cell lines are used to establish master cell banks for stockpiling the single cell progenitor clones expressing desired recombinant protein. Cells from the master cell bank are then used to create working cell banks that supply the cells that will be used for production. Single cell cloning is performed by aliquoting the amplified or unamplified cells into multi-well vessels, such as 96 well plates, which are maintained in a constant temperature culture, typically a physiological temperature such as 35° C.-37° C. typically 36° C.

Technologies are available to assist with single cell isolation from a population. These include, but are not limited to limiting dilution plating, microfluidic encapsulation, FACS-assisted cell sorting, colony picking in semi-solid media, single-cell printers, microfluidic wells or chips, including optifluidic technology, (Berkley Lights, Emeryville. CA). Only those colonies arising from a single cell are selected for further processing.

The stable pools generated during CLD workflow comprise a heterogeneous mix of cells which differ in the number and localization of integrated transgenes, have unique genetic and phenotypic characteristics, and/or are divergent in cell-specific productivity. These stably transfected pools are used for single cell cloning to generate clonally derived cultures that are subsequently screened for performance to identify those that have the desired qualities and characteristics, such as expression, productivity, growth, and/or other product quality attributes, including product-related impurity production. The transgene copy number can also be amplified using an inhibitor to the selection marker which the cells need to survive. This increases the copy number of the selection marker genes as well as transgenes integrated in the adjacent loci. In one embodiment the clonally derived culture may be further selected for desired expression, productivity, growth, and/or other desired product quality attributes of the expressed asymmetric multispecific antibody.

The clones are then scaled up using vessels with increasing size and are still maintained in a constant physiological temperature culture, 35° C.-37° C. typically 36° C. The vessels can include multi-well plates, for example, 96 well plates, 24 well plates, 6 well plates.

Once the cells have reached a desired cell number or density, clones are evaluated and ranked under conditions that mimic production conditions and are assessed for production and product qualities for the first time. The clones are transferred to suitable vessels, such as deep well plates, spin tubes, and/or agitated shake flasks, and are grown in suspension in a fed batch culture, incubated at a constant physiological temperature of 35° C.-37° C. typically 36° C. This is typically a 10-day process, with feeding and monitoring of culture conditions (e.g., pH, osmolality, lactate/glucose, etc.) every 2-3 days. Typically, at least 100 or more clones are screened. The ranking of pools and clones considers titer, specific productivity (qP), growth, viability, and product quality (PQ) profile, and the like. The specific product qualities that are used for the selection usually depend on the modality of the expressed protein and the qualities desired and/or required in the final drug product and/or during the production of the drug substance. These can include aggregate levels, charge distribution, clips, partial species, and post translational modifications. The top clones, typically 10 or less, are selected for further development.

The top clone candidates are typically cultured in small scale bioreactors (typically 3 L or 7 L) under some/all conditions that will be used during large scale production. For example, if one or more temperature shifts could occur during large scale production, these temperatures set points are incorporated during the small-scale bioreactor runs. This process is typically a 10-25 or more day process. In some embodiments the process is 15-20 days. In some embodiments the process is at least 15 days. The clones are again assessed and ranked based on productivity, growth, product quality attributes, and the like.

When this process was used to screen and select clonal cells expressing asymmetric multispecific antibodies it was observed that the product quality attributes of the proteins expressed by the clonal cells that were screened and selected at a constant physiological temperature (36° C.) changed when the cells were subjected to cell culture conditions that included a temperature shift (36° C. to 34° C. or 36° C. to 32.5° C.). Exposure to the lower culture temperatures resulted in an increase in the molar ratio of long heavy chain to short heavy chain. It is possible that the transcription of both the long and short heavy chains continues at the lower temperatures but at different rates, resulting in the skewed molar ratios observed. The shift in the ratio of the heavy chains impacted the type of product-related impurities that were formed, and these impurities impacted the product yield and activity, as well as the efficiency of the downstream purification operations.

The product quality of these asymmetric multispecific antibodies was influenced by the temperature during cell culture. To better understand this result, clones expressing asymmetric multispecific antibodies were exposed to one or more temperature set points during the initial screen and selection of clones based on product quality attributes. When temperature was included as a factor in the screening and selection of the clones it was possible to identify those clones that were able to maintain a more balanced molar ratio of long heavy chain to short heavy chain at different temperature set points. As such, the composition of the product related impurities changed, which improved downstream purification and improved the yield of the desired product, as well contributing to a more robust manufacturing process.

Temperature regime refers to the temperature(s) or temperature set points employed during cell culture. One or more temperature regimes may be used over the course of a cell culture operation. Most recombinant therapeutic proteins expressed by mammalian host cells. For encourage optimal growth of the cells, mammalian cell cultures are typically maintained at temperatures of 36° C. to 37° C. To increase production efficiency of the desired recombinant protein, less than optimal cell growth conditions may be desired to promote enhanced production. A lower culture temperature(s) may be used to slow or stop cell growth and to promote production of the desired recombinant antibody. Use of temperature shifts from growth phase optimal temperatures to production phase optimal temperatures are often employed in cell culture strategies. The growth phase may be conducted at a first temperature from about 28° C. to about 37° C., typically 36° C. to 37° C., and a production phase may conducted at a second temperature from about 28° C. to about 37° C., typically from about 30° C. to about 35° C., or from about 30° C. to about 34° C., preferably 32.5° C.

This modulation of temperature can be used throughout the duration of the cell culture to achieve the desired production objectives. Combinations of temperature shifts may also be used to go from a first growth phase, to a first production phase, to second growth phase, followed by a second production phase, and so on.

The invention provides for two or more temperature regimes. One embodiment of the invention comprises one or more single temperature regimes. In a single temperature regime, the culture is held at a single temperature for the duration of the culture. In one embodiment the temperature is from about 37° C. to about 28° C. In one embodiment the temperature is from about 36° C. to about 28° C. In one embodiment the temperature is from about 37° C. to about 29° C. In one embodiment the temperature is from about 36° C. to about 29° C. In one embodiment the temperature is from about 37° C. to about 30° C. In one embodiment the temperature is from about 36° C. to about 30° C. In one embodiment the temperature is from about 37° C. to about 31° C. In one embodiment the temperature is from about 36° C. to about 31° C. In one embodiment the temperature is from about 36° C. to about 31° C. In one embodiment the temperature is from about 37° C. to about 32° C. In one embodiment the temperature is from about 37° C. to about 32.5° C. In one embodiment the temperature is from about 36° C. to about 32° C. In one embodiment the temperature is from about 36° C. to about 32.5° C. In one embodiment the temperature is from about 37° C. to about 33° C. In one embodiment the temperature is from about 36° C. to about 33° C. In one embodiment the temperature is from about 37° C. to about 34° C. In one embodiment the temperature is from about 36° C. to about 34° C. In one embodiment the temperature is from about 37° C. to about 35° C. In one embodiment the temperature is from about 36° C. to about 35° C. In one embodiment the temperature is from about 37° C. to about 36° C. In one embodiment the temperature is from about 35° C. to about 28° C. In one embodiment the temperature is from about 35° C. to about 29° C. In one embodiment the temperature is from about 35° C. to about 30° C. In one embodiment the temperature is from about 35° C. to about 31° C. In one embodiment the temperature is from about 35° C. to about 32° C. In one embodiment the temperature is from about 35° C. to about 33° C. In one embodiment the temperature is from about 35° C. to about 34° C. In one embodiment the temperature is from about 34° C. to about 28° C. In one embodiment the temperature is from about 34° C. to about 29° C. In one embodiment the temperature is from about 34° C. to about 30° C. In one embodiment the temperature is from about 34° C. to about 31° C. In one embodiment the temperature is from about 34° C. to about 33° C. In one embodiment the temperature is from about 34° C. to about 28° C. In one embodiment the temperature is from about 33° C. to about 28° C. In one embodiment the temperature is from about 33° C. to about 29° C. In one embodiment the temperature is from about 33° C. to about 30° C. In one embodiment the temperature is from about 33° C. to about 31° C. In one embodiment the temperature is from about 33° C. to about 32° C. In one embodiment the temperature is from about 32° C. to about 28° C. In one embodiment the temperature is from about 32° C. to about 29° C. In one embodiment the temperature is from about 32° C. to about 30° C. In one embodiment the temperature is from about 32° C. to about 31° C. In one embodiment the temperature is from about 31° C. to about 28° C. In one embodiment the temperature is from about 31° C. to about 29° C. In one embodiment the temperature is from about 31° C. to about 30° C. In one embodiment the temperature is from about 30° C. to about 28° C. In one embodiment the temperature is from about 30° C. to about 29° C. In one embodiment the temperature is 28° C., 29°C., 30° C., 31° C., 32° C., 33° C., 34° C., 35° C., 36° C. or 37° C. In one embodiment the temperature is 32.5° C.

One embodiment of the invention comprises one or more temperature regimes comprising at least one temperature shift. In one embodiment the temperature shift is from a first temperature to a second temperature that is higher or lower than the first temperature. In one embodiment the first temperature is from about 37° C. to about 28° C. In one embodiment the first temperature is from about 36° C. to about 28° C. In one embodiment the first temperature is from about 37° C. to about 29° C. In one embodiment the first temperature is from about 36° C. to about 29° C. In one embodiment the first temperature is from about 37° C. to about 30° C. In one embodiment the first temperature is from about 36° C. to about 30° C. In one embodiment the first temperature is from about 37° C. to about 31° C. In one embodiment the first temperature is from about 36° C. to about 31° C. In one embodiment the first temperature is from about 36° C. to about 31° C. In one embodiment the first temperature is from about 37° C. to about 32° C. In one embodiment the first temperature is from about 37° C. to about 32.5° C. In one embodiment the first temperature is from about 36° C. to about 32° C. In one embodiment the first temperature is from about 36° C. to about 32.5° C. In one embodiment the first temperature is from about 37° C. to about 33° C. In one embodiment the first temperature is from about 36° C. to about 33° C. In one embodiment the first temperature is from about 37° C. to about 34° C. In one embodiment the first temperature is from about 36° C. to about 34° C. In one embodiment the first temperature is from about 37° C. to about 35° C. In one embodiment the first temperature is from about 36° C. to about 35° C. In one embodiment the first temperature is from about 35° C. to about 28° C. In one embodiment the first temperature is from about 35° C. to about 29° C. In one embodiment the first temperature is from about 35° C. to about 30° C. In one embodiment the first temperature is from about 35° C. to about 31° C. In one embodiment the first temperature is from about 35° C. to about 32° C. In one embodiment the first temperature is from about 35° C. to about 32.5° C. In one embodiment the first temperature is from about 35° C. to about 33° C. In one embodiment the first temperature is from about 35° C. to about 34° C. In one embodiment the first temperature is from about 34° C. to about 28° C. In one embodiment the first temperature is from about 34° C. to about 29° C. In one embodiment the first temperature is from about 34° C. to about 30° C. In one embodiment the first temperature is from about 34° C. to about 31° C. In one embodiment the first temperature is from about 34° C. to about 32° C. In one embodiment the first temperature is from about 34° C. to about 32.5° C. In one embodiment the first temperature is from about 34° C. to about 28° C. In one embodiment the first temperature is from about 33° C. to about 28° C. In one embodiment the first temperature is from about 33° C. to about 29° C. In one embodiment the first temperature is from about 33° C. to about 30° C. In one embodiment the first temperature is from about 33° C. to about 31° C. In one embodiment the first temperature is from about 33° C. to about 32° C. In one embodiment the first temperature is from about 33° C. to about 32.5° C. In one embodiment the first temperature is from about 32° C. to about 28° C. In one embodiment the first temperature is from about 32° C. to about 29° C. In one embodiment the first temperature is from about 32° C. to about 30° C. In one embodiment the first temperature is from about 32.5° C. to about 28° C. In one embodiment the first temperature is from about 32.5° C. to about 29° C. In one embodiment the first temperature is from about 32.5° C. to about 30° C. In one embodiment the first temperature is from about 32.5° C. to about 31° C. In one embodiment the first temperature is from about 32.5° C. to about 32° C. In one embodiment the first temperature is from about 32.5° C. to about 33° C. In one embodiment the first temperature is from about 32.5° C. to about 34° C. In one embodiment the first temperature is from about 32.5° C. to about 35° C. In one embodiment the first temperature is from about 31° C. to about 28° C. In one embodiment the first temperature is from about 31° C. to about 29° C. In one embodiment the first temperature is from about 31° C. to about 30° C. In one embodiment the first temperature is from about 30° C. to about 28° C. In one embodiment the first temperature is from about 30° C. to about 29° C. In one embodiment the first temperature is 28° C., 29° C., 30° C., 31° C., 32° C., 33° C., 34° C., 35° C., 36° C. or 37° C. In one embodiment the first temperature is 32.5° C.

In one embodiment the second temperature is about 1° C. to 9° C. lower than the first temperature. In one embodiment the second temperature is about 2° C. to 9° C. lower than the first temperature. In one embodiment the second temperature is about 3° C. to 9° C. lower than the first temperature. In one embodiment the second temperature is about 4° C. to 9° C. lower than the first temperature. In one embodiment the second temperature is about 5° C. to 9° C. lower than the first temperature. In one embodiment the second temperature is about 6° C. to 9° C. lower than the first temperature. In one embodiment the second temperature is about 7° C. to 9° C. lower than the first temperature. In one embodiment the second temperature is about 8° C. to 9° C. lower than the first temperature. In one embodiment the second temperature is about 1° C. to 8° C. lower than the first temperature. In one embodiment the second temperature is about 2° C. to 8° C. lower than the first temperature. In one embodiment the second temperature is about 3° C. to 8° C. lower than the first temperature. In one embodiment the second temperature is about 4° C. to 8° C. lower than the first temperature. In one embodiment the second temperature is about 5° C. to 8° C. lower than the first temperature. In one embodiment the second temperature is about 6° C. to 8° C. lower than the first temperature. In one embodiment the second temperature is about 7° C. to 8° C. lower than the first temperature. In one embodiment the second temperature is about 1° C. to 7° C. lower than the first temperature. In one embodiment the second temperature is about 2° C. to 7° C. lower than the first temperature. In one embodiment the second temperature is about 3° C. to 7° C. lower than the first temperature. In one embodiment the second temperature is about 4° C. to 7° C. lower than the first temperature.

In one embodiment the second temperature is about 5° C. to 7° C. lower than the first temperature. In one embodiment the second temperature is about 6° C. to 7° C. lower than the first temperature. In one embodiment the second temperature is about 1° C. to 6° C. lower than the first temperature. In one embodiment the second temperature is about 2° C. to 6° C. lower than the first temperature. In one embodiment the second temperature is about 3° C. to 6° C. lower than the first temperature. In one embodiment the second temperature is about 4° C. to 6° C. lower than the first temperature. In one embodiment the second temperature is about 5° C. to 6° C. lower than the first temperature. In one embodiment the second temperature is about 1° C. to 5° C. lower than the first temperature. In one embodiment the second temperature is about 2° C. to 5° C. lower than the first temperature. In one embodiment the second temperature is about 3° C. to 5° C. lower than the first temperature. In one embodiment the second temperature is about 4° C. to 5° C. lower than the first temperature. In one embodiment the second temperature is about 1° C. to 4° C. lower than the first temperature. In one embodiment the second temperature is about 2° C. to 4° C. lower than the first temperature. In one embodiment the second temperature is about 3° C. to 4° C. lower than the first temperature. In one embodiment the second temperature is about 1° C. to 3° C. lower than the first temperature. In one embodiment the second temperature is about 2° C. to 3° C. lower than the first temperature. In one embodiment the second temperature is about 2.5° C. to 3.5° C. lower than the first temperature. In one embodiment the second temperature is about 1° C. to 2° C. lower than the first temperature.

In one embodiment the second temperature is from about 37° C. to about 28° C. In one embodiment the second temperature is from about 36° C. to about 28° C. In one embodiment the second temperature is from about 37° C. to about 29° C. In one embodiment the second temperature is from about 36° C. to about 29° C. In one embodiment the second temperature is from about 37° C. to about 30° C. In one embodiment the second temperature is from about 36° C. to about 30° C. In one embodiment the second temperature is from about 37° C. to about 31° C. In one embodiment the second temperature is from about 36° C. to about 31° C. In one embodiment the second temperature is from about 36° C. to about 31° C. In one embodiment the second temperature is from about 37° C. to about 32° C. In one embodiment the second temperature is from about 37° C. to about 32.5° C. In one embodiment the second temperature is from about 36° C. to about 32° C. In one embodiment the second temperature is from about 36° C. to about 32.5° C. In one embodiment the second temperature is from about 37° C. to about 33° C. In one embodiment the second temperature is from about 36° C. to about 33° C. In one embodiment the second temperature is from about 37° C. to about 34° C. In one embodiment the second temperature is from about 36° C. to about 34° C. In one embodiment the second temperature is from about 37° C. to about 35° C. In one embodiment the second temperature is from about 36° C. to about 35° C. In one embodiment the second temperature is from about 35° C. to about 28° C. In one embodiment the second temperature is from about 35° C. to about 29° C. In one embodiment the second temperature is from about 35° C. to about 30° C. In one embodiment the second temperature is from about 35° C. to about 31° C. In one embodiment the second temperature is from about 35° C. to about 32° C. In one embodiment the second temperature is from about 35° C. to about 32.5° C. In one embodiment the second temperature is from about 35° C. to about 33° C. In one embodiment the second temperature is from about 35° C. to about 34° C. In one embodiment the second temperature is from about 34° C. to about 28° C. In one embodiment the second temperature is from about 34° C. to about 29° C. In one embodiment the second temperature is from about 34° C. to about 30° C. In one embodiment the second temperature is from about 34° C. to about 31° C. In one embodiment the second temperature is from about 34∪ C. to about 32° C. In one embodiment the second temperature is from about 34° C. to about 32.5° C. In one embodiment the second temperature is from about 34° C. to about 28° C. In one embodiment the second temperature is from about 33°0 C. to about 28° C. In one embodiment the second temperature is from about 33° C. to about 29° C. In one embodiment the second temperature is from about 33° C. to about 30° C. In one embodiment the second temperature is from about 33° C. to about 31° C. In one embodiment the second temperature is from about 33° C. to about 32° C. In one embodiment the second temperature is from about 33° C. to about 32.5° C. In one embodiment the second temperature is from about 32° C. to about 28° C. In one embodiment the second temperature is from about 32° C. to about 29° C. In one embodiment the second temperature is from about 32° C. to about 30° C. In one embodiment the second temperature is from about 32.5° C. to about 28° C. In one embodiment the second temperature is from about 32.5° C. to about 29° C. In one embodiment the second temperature is from about 32.5° C. to about 30° C. In one embodiment the second temperature is from about 32.5° C. to about 31° C. In one embodiment the second temperature is from about 31° C. to about 28° C. In one embodiment the second temperature is from about 31° C. to about 29° C. In one embodiment the second temperature is from about 31° C. to about 30° C. In one embodiment the second temperature is from about 30° C. to about 28° C. In one embodiment the second temperature is from about 30° C. to about 29° C. In one embodiment the second temperature is 28° C., 29° C., 30° C., 31° C., 32° C., 33° C., 34° C., 35° C., 36° C. or 37° C. In one embodiment the second temperature is 32.5° C.

In one embodiment the first temperature regime is a single temperature from 36° C. to 37° C. and the second temperature regime comprises at least one temperature shift from a first temperature of 36° C. to 37° C. to a second temperature of 28° C. to 35° C.

“Asymmetric multispecific antibody” include asymmetric proteins that are recombinantly engineered to bind, neutralize, and/or interact specifically with at least two different antigens or targets, or at least two different epitopes on the same antigen or target. In one embodiment, asymmetric multispecific antibodies can be engineered to target immune effectors, to stimulate or trigger immune responses, to carry cytotoxic agents to tumors or infectious agents. These asymmetric multispecific antibodies have been found useful for a variety of applications such as in cancer immunotherapy by redirecting immune effector cells to tumor cells, modifying cell signaling by blocking signaling pathways, targeting tumor angiogenesis, blocking cytokines, and as pre-targeted delivery vehicles for drugs, such as delivery of chemotherapeutic agents, radiolabels (to improve detection sensitivity) and nanoparticles (directed to specific cells/tissues, such as cancer cells).

The asymmetric multispecific antibodies can be of scientific and/or commercial interest. Asymmetric multispecific antibodies can be produced in various ways, most commonly by recombinant animal cell lines using cell culture methods. The asymmetric multispecific antibodies may be produced intracellularly or secreted into the culture medium from which it can be recovered and/or collected and may be referred to as “recombinant asymmetric multispecific antibodies”. The term “isolated recombinant asymmetric multispecific antibodies” refer to an asymmetric multispecific antibodies that that have been purified away from proteins, polypeptides. DNA, and/or other contaminants or impurities that would interfere with its therapeutic, diagnostic, prophylactic, research, or other use. Asymmetric multispecific antibodies of interest include, among others, those that exert a therapeutic effect by binding two or more targets, particularly targets among those listed herein, including targets derived therefrom, targets related thereto, and modifications thereof. Asymmetric multispecific antibodies may have two or more heavy chains that differ in length from each other, for example, having a long heavy chain and a short heavy chain. In a related embodiment, the asymmetric multispecific antibody is an asymmetric bispecific antibody. Various bispecific antibody formats that include asymmetric bispecific antibodies are known in the art (Spiess et al., Mol Immunol 67, 95-106, 2015; Sedykh et al., Drug Design, Development and Therapy 18(12), 195-208, 2018; Fan et al., J Hematol & Oncology 8:130-143, 2015;); Williams et al., Chapter 41 Process Design for Bispecific Antibodies in Biopharmaceutical Processing Development, Design and Implementation of Manufacturing Processes, Jagschies et al., eds., 2018, pages 837-855; Seimetz et al., Cancer Treat Rev 36(6) 458-67, 2010; Shulka and Norman, Chapter 26 Downstream Processing of Fc Fusion Proteins, Bispecific Antibodies, and Antibody-Drug Conjugates, in Process Scale Purification of Antibodies Second Edition, Uwe Gottswchalk editor, p 559-594, John Wiley & Sons, 2017; Moore et al., MAbs 3:6, 546-557, 2011.

Asymmetrical multispecific antibodies also include those having an odd number of active components having unique peptide and/or protein sequences that collectively result in an asymmetrical multispecific antibody with specificity for two or more targets. Antibodies include human, humanized, chimeric, multi-specific, monoclonal, polyclonal, bispecific, and oligomers or antigen binding fragments thereof. Antibodies also include both glycosylated and non-glycosylated immunoglobulins of any isotype or subclass, such as lgG1-, lgG2-lgG3- or lgG4-type. Also included are antibodies having an antigen binding fragment or region, particularly antibody antigen binding fragments that compete with the intact antibody for specific binding. Also included are peptibodies, antibody derivatives, antibody analogs, fusion proteins (and proteins made using Xmab® technology, bispecific T cell engagers having extensions, such as half-life extensions, for example BiTE® molecules.

Also included are modified asymmetric multispecific antibodies, such as antibodies modified chemically by a non-covalent bond, covalent bond, or both a covalent and non-covalent bond. Also included are antibodies further comprising one or more post-translational modifications which may be made by cellular modification systems or modifications introduced ex vivo by enzymatic and/or chemical methods or introduced in other ways. In particular, antibodies modified using such methods to attach one or more proteins of interest to the antibody.

In some embodiments, asymmetric multispecific antibodies bind, neutralize and/or interact specifically to one or more CD proteins. HER receptor family proteins, cell adhesion molecules, growth factors, nerve growth factors, fibroblast growth factors, transforming growth factors (TGF), insulin-like growth factors, osteoinductive factors, insulin and insulin-related proteins, coagulation and coagulation-related proteins, colony stimulating factors (CSFs), other blood and serum proteins blood group antigens; receptors, receptor-associated proteins, growth hormones, growth hormone receptors, T-cell receptors; neurotrophic factors, neurotrophins, relaxins, interferons, interleukins, viral antigens, lipoproteins, integrins, rheumatoid factors, immunotoxins, surface membrane proteins, transport proteins, homing receptors, addressins, regulatory proteins, and immunoadhesins.

In some embodiments asymmetric multispecific antibodies bind, neutralize and/or interact with one or more of the following, alone or in any combination: CD proteins including but not limited to CD3, CD4, CD5, CD7, CD8, CD19, CD20, CD22, CD25, CD30, CD33, CD34, CD38, CD40, CD70, CD123, CD133, CD138, CD17,. and CD174, HER receptor family proteins, including, for instance, HER2, HER3, HER4, and the EGF receptor, EGFRvIII, cell adhesion molecules, for example, LFA-1, Mol, p 150, 95, VLA-4, ICAM-1, VCAM, and alpha v/beta 3 integrin, growth factors, including but not limited to, for example, vascular endothelial growth factor (“VEGF”); VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoictin (EPO), nerve growth factor, such as NGF-beta, platelet-derived growth factor (PDGF), fibroblast growth factors, including, for instance, aFGF and bFGF, epidermal growth factor (EGF), Cripto, transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors, insulins and insulin-related proteins, including but not limited to insulin, insulin A-chain, insulin B-chain, proinsulin, and insulin-like growth factor binding proteins; (coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs), including the following, among others, M-CSF, GM-CSF, and G-CSF, other blood and serum proteins, including but not limited to albumin, IgE, and blood group antigens, receptors and receptor-associated proteins, including, for example, flk2/flt3 receptor, obesity (OB) receptor, growth hormone receptors, and T-cell receptors; neurotrophic factors, including but not limited to, bone-derived neurotrophic factor (BDNF) and neurotrophin-3, -4, -5, or -6 (NT-3, NT-4, NT-5, or NT-6); relaxin A-chain, relaxin B-chain, and prorelaxin, interferon, including for example, interferon-alpha, -beta, and -gamma, interleukins (ILs), e.g., IL-1 to IL-10, IL-12, IL-15, IL-17, IL-23, IL-12/IL-23, IL-2Ra, IL1-R1, IL-6 receptor, IL-4 receptor and/or IL-13 to the receptor, IL-13RA2, or IL-17 receptor, IL-1RAP, viral antigens, including but not limited to, an AIDS envelope viral antigen, lipoproteins, calcitonin, glucagon, atrial natriuretic factor, lung surfactant, tumor necrosis factor-alpha and -beta, enkephalinase, BCMA, IgKappa, ROR-1, ERBB2, mesothelin, RANTES (regulated on activation normally T-cell expressed and secreted). mouse gonadotropin-associated peptide, Dnase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, addressins, regulatory proteins, immunoadhesins, antigen-binding proteins, somatropin, CTGF, CTLA4, cotaxin-1, MUC1, CEA, c-MET, Claudin-18, GPC-3, EPHA2, FPA, LMP1, MG7, NY-ESO-1, PSCA, ganglioside GD2, glanglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, NKG2D-1, programmed cell death protein 1 and ligand, PD1 and PDL1, mannose receptor/hCGβ, hepatitis-C virus, mesothelin dsFv[PE38 conjugate, Legionella pneumophila (lly), IFN gamma, interferon gamma induced protein 10 (IP10), IFNAR, TALL-1, TNFα, TL1A, thymic stromal lymphopoietin (TSLP), proprotein convertase subtilisin/Kexin Type 9 (PCSK9), stem cell factors, Flt-3, calcitonin gene-related peptide (CGRP), OX40L, α4β7, platelet specific (platelet glycoprotein Iib/IIIb (PAC-1), transforming growth factor beta (TFGβ), STEAP1, Zona pellucida sperm-binding protein 3 (ZP-3), TWEAK, platelet derived growth factor receptor alpha (PDGFRα), sclerostin, and biologically active fragments or variants of any of the foregoing. In some embodiments the asymmetric multispecific antibody comprises a mutein attached to an antibody or immunoglobulin, for example the IL-2 variant immunocytokine. Examples are CEA-IL-2v, (cergutuzumab amunaleukin), where an IL-2v moiety is fused to one heavy chain of the Fc part of the antibody, Schneider et al., 2019. Biotechnology Bioengineering 116:2503-2513; and Anti-FAP-IL-2 (fibroblast activation protein (FAP) targeted interleukin-2 variant (IL-2v), Soerensen M M, et al. Safety, PK/PD, and anti-tumor activity of RO6874281. an engineered variant of interleukin-2 (IL-2v) targeted to tumor-associated fibroblasts via binding to fibroblast activation protein (FAP), Nicolini V. et al. Combining CEA-IL2v and FAP-IL2v immunocytokines with PD-L1 checkpoint blockade. Annual Meeting of the American Association for Cancer Research; New Orleans: 2016.

In some aspects the asymmetric multispecific antibody is a bifunctional fusion protein. In some aspects the bifunctional fusion protein comprises an IL-21 mutein. In some aspects the bifunctional fusion protein comprises a conjugate of an IL-21 mutein linked to the C-terminus of one of the two antibody heavy chains of an anti-PD-1 antibody. The IL-21-anti-PD-1 conjugate comprises a “short heavy chain” (an anti-PD1 heavy chain) and a “long heavy chain” (an anti-PD1 heavy chain linked to the IL-21 mutein). Such antibodies arc described in WO2019/028316, incorporated by reference in its entirety.

Interleukin-21 (IL-21) is a cytokine expressed by T cells, B cells, NK cells and myeloid cells, and regulates the activity of both innate and adaptive immune cells and improves T cell survival and effector function. IL-21 has a four-helix bundle structure and exists as a monomer. In some embodiments the asymmetric proteins comprise IL-21 muteins comprising at least one amino acid substitution, relative to the wild-type IL-21 amino acid sequence, which is provided herein as SEQ ID NO: 1. In exemplary aspects, the IL-21 mutein comprises an amino acid sequence of SEQ ID NO: 2, wherein X is any amino acid, and wherein the IL-21 mutein amino acid sequence differs from the amino acid sequence of human IL-21 (SEQ ID NO: 1) by at least 1 amino acid. In various aspects, the IL-21 mutein comprises an amino acid sequence which differs from the amino acid sequence of human IL-21 (SEQ ID NO: 1) by 3, 4, 5, 6 or 7 amino acids. In various aspects, the IL-21 mutein comprises an amino acid sequence which differs from the amino acid sequence of human IL-21 (SEQ ID NO: 1) by 1 or 2 amino acids. In exemplary aspects, the IL-21 mutein comprises an amino acid substitution with an amino acid at the position according to Table 1.

TABLE 1
Amino acid substitutions

| Amino Acid | |
|---|---|
| position of | Amino Acid |
| SEQ ID NO: 1 | (in single letter code) |
| 5 | A, D, E, G, H, I, K, L, M, N, Q, |
| | S, T, V, or Y |
| 8 | A, D, E, N, S, T, V, or Y |
| 9 | A, D, E, G, H, I, K, L, M, N, Q, |
| | S, T, V, or Y |
| 11 | D or S |
| 12 | A, D, E, N, S, T, or V |
| 13 | D |
| 14 | A, D, or S |
| 15 | A, E, I, M, N, Q, S, T, or V |
| 16 | D or E |
| 19 | D |
| 23 | D |
| 65 | D, G, or P |
| 66 | D, G, or P |
| 68 | Q |
| 69 | D, G, or P |
| 70 | E, G, P, or Y |
| 71 | L |
| 72 | D, G, or P |
| 73 | A, D, E, G, H, I, N, P, Q, S, or V |
| 75 | D, G, or P |
| 76 | A, D, E, G, H, I, K, L, M, N, P, |
| | Q, S, T, V, or Y |
| 77 | D, G, or P |
| 78 | D |
| 79 | D |
| 80 | G, or P |
| 109 | K |
| 110 | D |
| 112 | D |
| 113 | K |
| 116 | A, D, E, I, K, L, M, N, S, T, or V |
| 117 | D |
| 119 | A, D, E, M, N, Q, S, or T |
| 120 | D |
| 123 | D |

In various aspects, the asymmetric multispecific antibody comprises an IL-21 mutein comprising one amino acid substitution relative to SEQ ID NO: 1 and optionally comprises one of SEQ ID NOs: 3-198, 249-254, and 283. In some embodiments the asymmetric multispecific antibody comprises an IL-21 mutein comprising two amino acid substitutions relative to SEQ ID NO: 1 and optionally comprising one of SEQ ID NOs: 199-248, and 255. In some embodiments the asymmetric multispecific antibody comprises an IL-21 mutein comprising amino acid substitutions at any two of positions 5, 9, 73, and 76 of SEQ ID NO: 1. In one embodiment amino acid substitutions are selected from: A, E, or Q at position 5, E or A at position 9, A or Q at position 73, and A, D, or E, at position 76. In one embodiment, the amino acid substitutions are at positions 5 and 73 of SEQ ID NO: 1. In one embodiment, the amino acid substitutions are at positions 5 and 76 of SEQ ID NO: 1. In one embodiment, the amino acid substitutions are at positions 9 and 73 of SEQ ID NO: 1. In one embodiment, the amino acid substitutions are at positions 9 and 76 of SEQ ID NO:1. In one embodiment the IL-21 mutein comprises the amino acid sequence of any of SEQ ID NOs: 233-245. In one embodiment, a single IL-21 mutein of SEQ ID NO: 233, 234, 235, 236, 237, 238, 239, 240, 241, 242, 243, 244, or 245 is attached to the Fc of the anti-PD-1 antibody. In one embodiment, a single IL-21 mutein of SEQ ID NO: 233, 234, 235, 236, 237, 238, 239, 240, 241, 242, 243, 244, or 245 is attached to the Fc of the anti-PD-1 antibody. In one embodiment the IL-21 mutein comprises the amino acid sequence of SEQ ID NO: 233. In one embodiment the IL-21 mutein comprises the amino acid sequence of SEQ ID NO: 234. In one embodiment the IL-21 mutein comprises the amino acid sequence of SEQ ID NO: 235. In one embodiment the IL-21 mutein comprises the amino acid sequence of SEQ ID NO: 236. In one embodiment the IL-21 mutein comprises the amino acid sequence of SEQ ID NO: 237. In one embodiment the IL-21 mutein comprises the amino acid sequence of SEQ ID NO: 238. In one embodiment the IL-21 mutein comprises the amino acid sequence of SEQ ID NO: 239. In one embodiment the IL-21 mutein comprises the amino acid sequence of SEQ ID NO: 240. In one embodiment the IL-21 mutein comprises the amino acid sequence of SEQ ID NO: 241. In one embodiment the IL-21 mutein comprises the amino acid sequence of SEQ ID NO: 242. In one embodiment the IL-21 mutein comprises the amino acid sequence of SEQ ID NO: 243. In one embodiment the IL-21mutein comprises the amino acid sequence of SEQ ID NO: 244. In one embodiment the IL-21 mutein comprises the amino acid sequence of SEQ ID NO: 245.

In some embodiments the asymmetric multispecific antibody comprises a conjugate of a mutein and an antibody or immunoglobulin. In some embodiments the conjugate comprises an IL-21 mutein. In some embodiments the conjugate comprises an IL-21 mutein attached to an antibody or immunoglobulin.

In some embodiments the asymmetric multispecific antibody comprises an IL-21 mutein attached via non-covalent or covalent bonding to an antibody or immunoglobulin, e.g., peptide bonds, disulfide bonds, and the like, or via physical forces, such as electrostatic, hydrogen, ionic, van der Waals, or hydrophobic or hydrophilic interactions. A variety of non-covalent coupling systems may be used, including, e.g., biotin-avidin, ligand/receptor, enzyme/substrate, nucleic acid/nucleic acid binding protein, lipid/lipid binding protein, cellular adhesion molecule partners; or any binding partners or fragments thereof which have affinity for each other. In some embodiments the attachment may be via direct covalent linkage by reacting targeted amino acid residues of the IL-21 mutein with an organic derivatizing agent that is capable of reacting with selected side chains or the N- or C-terminal residues of these targeted amino acids.

In some embodiments the attachment between the IL-21 mutein and the antibody or immunoglobulin may be via a linker. In some embodiments, the linker comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker are selected from the group consisting of carbon (C), oxygen (O), nitrogen (N), and sulfur (S). Chain atoms and linkers may be selected according to their expected solubility (hydrophilicity) so as to provide a more soluble conjugate. In some embodiments, the linker provides a functional group that is subject to cleavage by an enzyme or other catalyst or hydrolytic conditions found in the target tissue or organ or cell. In some embodiments, the length of the linker is long enough to reduce the potential for steric hindrance. If the linker is a covalent bond or a peptidyl bond and the conjugate is a polypeptide, the entire conjugate can be a fusion protein. Such peptidyl linkers may be any length. Exemplary peptidyl linkers are from about 1 to 50 amino acids in length, 5 to 50, 3 to 5, 5 to 10, 5 to 15, or 10 to 30 amino acids in length, and are flexible or rigid. In some embodiments, the linker is a peptide comprising about 2 to about 20 amino acids. In some embodiments. the linker is a peptide comprising about 2 to about 15 amino acid, about 2 to about 10 amino acids, or about 2 to about 5 amino acids. Suitable peptide linkers are known in the art. See, e.g., Chen et al., Adv Drug Delivery Reviews 65(10): 1357-1369 (2013); Arai et al., Protein Eng Des Sel 14(8): 529-532 (2001); and Wriggers et al, Curr Trends in Peptide Science 80(6): 736-746 (2005). In one embodiment, the linker is a peptide comprising the amino acid sequence GGGGS (SEQ ID NO: 262).

The asymmetric multispecific antibody comprises an IL-21 mutein directly attached to an Fc of an antibody. Alternatively, the IL-21 mutein is attached to the Fc of the antibody via a linker, such as a peptide comprising the amino acid sequence of SEQ ID NO: 262. In various instances, the asymmetric multispecific antibody comprises a single IL-21 mutein, wherein said single IL-21 mutein is linked to the C-terminus of one of the two antibody heavy chains.

In various aspects, the antibody heavy chains comprise one or more amino acid modifications, relative to the naturally occurring counterpart, in order to improve half-life/stability or to render the antibody more suitable for expression/manufacturability (e.g., as an asymmetric multispecific antibody with the IL-21 mutein).

In exemplary instances, the antibody heavy chains are designed to prevent or reduce interaction between the antibody and Fc receptors. In exemplary instances, the antibody is a Stable Effector Functionless (SEFL) antibody comprising a constant region that lacks the ability to interact with Fcγ receptors. SEFL antibodies are known in the art. See, e.g., Liu et al., J Biol Chem 292; 1876-1883 (2016); and Jacobsen et al., J. Biol. Chem. 292; 1865-1875 (2017). In exemplary aspects, the SEFL antibody comprises one or more of the following mutations, numbered according to the EU system: L242C, A287C, R292C, N297G, V302C, L306C, and/or K334C. In exemplary aspects, the SEFL antibody comprises N297G. In exemplary aspects, the SEFL antibody comprises A287C, N297G, and L306C. In other exemplary aspects, the SEFL antibody comprises R292C, N297G, and V302C (i.e., SEFL2-2).

The antibody may comprise other half-life extension (HLE) modifications. In exemplary instances, the HLE modification occurs in the heavy chain constant region and comprises one or more of the following mutations, numbered according to the EU system: M252Y, S254T, and T256E. In exemplary instances, the antibody comprises one or two of M252Y, S254T, and T256E. In exemplary instances, the antibody comprises all three of M252Y, S254T, and T256E. In exemplary aspects, the heavy chain constant region comprises an amino acid sequence of SEQ ID NO: 545 or SEQ ID NO: 547 or SEQ ID NO: 549. In exemplary instances, the HLE modification occurs in the heavy chain constant region and comprises one or more of the following mutations, numbered according to the EU system: L309D, Q311H, and N434S. In exemplary instances, the antibody comprises one, two or all three of L309D, Q311H, and N434S. In exemplary instances, the antibody comprises all three of L309D, Q311H, and N434S. In exemplary aspects, the heavy chain constant region comprises an amino acid sequence of SEQ ID NO: 544 or SEQ ID NO: 546 or SEQ ID NO: 548.

In exemplary aspects, the antibody comprises SEFL2-2 modifications and HLE modifications. In some instances, the HLE modifications comprise one or two or all three of M252Y, S254T, and T256E. In exemplary aspects, the heavy chain constant region comprises an amino acid sequence of SEQ ID NO: 551 or SEQ ID NO: 553 or SEQ ID NO: 555. In some instances, the HLE modifications comprise one or two or all three of L309D, Q311H, and N434S. In exemplary aspects, the heavy chain constant region comprises an amino acid sequence of SEQ ID NO: 550 or SEQ ID NO: 552 or SEQ ID NO: 554. In exemplary aspects, the heavy chain additionally comprises charge pair mutations as described below.

In general, the C-terminal lysine of an antibody undergoes cleavage by carboxypeptidase during expression. A heavy chain constant region lacking the C-terminal Lys advantageously prevents carboxypeptidase to act on the heavy chain of the antibody. In exemplary aspects, the antibody comprises a heavy chain constant region lacking the C-terminal Lys and further comprises a linker, such as a peptide comprising the amino acid sequence of SEQ ID NO: 262.

In exemplary aspects, when the asymmetric multispecific antibody comprises only one IL-21 mutein, the Fc of the antibody comprises modifications designed to drive heterodimerization of the two heavy chains (one heavy chain fused to the IL-21 mutein and one heavy chain lacking the IL-21 mutein). Such modifications include Fc mutations such as knobs-into-holes, DuoBodies, Azymetric, charge pair, HA-TF, SEEDbody, and modifications with differential protein A affinity. See, e.g., Spiess et al., Molecular Immunology, 67(2, Part A), 2015, pp. 95-106. Knobs-into-holes mutations include T366W in the first heavy chain, and T366S, L368A, and/or Y407V in the second heavy chain. See, e.g., Ridgway et al., Protein Eng., 9 (1996), pp. 617-621; and Atwell et al., J. Mol. Biol., 270 (1997), pp. 26-35. DuoBody mutations include F405L in the first heavy chain and K409R in the second heavy chain. See, e.g., Labrijn et al., Proc. Natl. Acad. Sci. U.S.A., 110 (2013), pp. 5145-5150. Azymetric mutations include T350V, L351Y, F405A, and/or Y407V in the first heavy chain, and T350V, T366L, K392L, and/or T394W in the second heavy chain. See, e.g.. Von Kreudenstein et al., mAbs, 5 (2013), pp. 646-654. HA-TF mutations include S364H and/or F405A in the first heavy chain, and Y349T and/or T394F in the second heavy chain. See, e.g., Moore et al., mAbs, 3 (2011), pp. 546-557. SEEDbody mutations include IgG/A chimera mutations in the first heavy chain and IgG/A chimera mutations in the second heavy chain, See, e.g., Davis et al., Protein Eng. Des. Sel., 23 (2010), pp. 195-202. Differential protein A affinity mutations include H435R in one heavy chain and no mutations in the other heavy chain. See, e.g., U.S. Pat. No. 8,586,713.

In a particular example, the mutations are charge pair mutations. The following are examples of such charge pair mutations, numbered according to the EU system. Charge pair mutations include K409D in the first heavy chain and D399K in the second heavy chain; K392D in the first heavy chain and E356K in the second heavy chain; or both K409D and K392D in the first heavy chain and both D399K and E356K in the second heavy chain (the latter denoted as “V1” herein). See, e.g., Gunasekaran et al., J Biol Chem 285: 19637-19646 (2010). In another particular example, the charge pair mutations include K439D, K392D, and K409D in the first heavy chain; and E356K and D399K in the second heavy chain (denoted as “V103” herein). In yet another particular example, the charge pair mutations include K360E, K370E, K392E, and K409D in the first heavy chain; and E357K and D399K in the second heavy chain (denoted as “V131” herein). Charge pair mutations may also include K370D in the first heavy chain and E357K in the second heavy chain; or all three of K409D, K392D, and K370D in the first heavy chain and all three of D399K, E357K, and E356K in the second heavy chain (the latter denoted as “V4” herein). Additional charge pair mutations also include D221E. P228E, and/or L368E in the first heavy chain and D221R, P228R, and/or K409R in the second heavy chain. See, e.g., Strop et al., J. Mol. Biol., 420 (2012), pp. 204-219.

In embodiments where the asymmetric multispecific antibody comprises only one IL-21 mutein (i.e., the asymmetric multispecific antibody comprises an IL-21 mutein monomer) and the heavy chain contains the V1 charge pair mutations, the IL-21 mutein may be attached to the heavy chain containing the K409D and K392D mutations (e.g., the IL-21 mutein is attached to a heavy chain comprising SEQ ID NO: 294, 296, or 298), or the heavy chain containing the D399K and E356K mutations (e.g., the IL-21 mutein is attached to a heavy chain comprising SEQ ID NO: 295, 297, or 299. In a specific embodiment, the IL-21 mutein is attached to the heavy chain containing the D399K and E356K mutations.

In embodiments where the asymmetric multispecific antibody comprises only one IL-21 mutein (i.e., the asymmetric multispecific antibody comprises an IL-21 mutein monomer) and the heavy chain contains the V4 charge pair mutations, the IL-21 mutein may be attached to the heavy chain containing the K409D, K392D, and K370D mutations (e.g., the IL-21 mutein is attached to a heavy chain comprising SEQ ID NO: 288, 290, or 292), or the heavy chain containing the D399K, E357K, and E356K mutations (e.g., the IL-21 mutein is attached to a heavy chain comprising SEQ ID NO: 289, 291, or 293). In a specific embodiment, the IL-21 mutein is attached to the heavy chain containing the D399K, E357K, and E356K mutations.

In embodiments where the asymmetric multispecific antibody comprises only one IL-21 mutein (i.e., the asymmetric multispecific antibody comprises an IL-21 mutein monomer) and the heavy chain contains the V103 charge pair mutations, the IL-21 mutein may be attached to the heavy chain containing the K439D, K392D, and K409D mutations (e.g., the IL-21 mutein is attached to a heavy chain comprising SEQ ID NO: 472, 474, or 476), or the heavy chain containing the E356K and D399K mutations (e.g., the IL-21 mutein is attached to a heavy chain comprising SEQ ID NO: 473, 475, or 477). In a specific embodiment, the IL-21 mutein is attached to the heavy chain containing the E356K and D399K mutations.

In embodiments where the asymmetric multispecific antibody comprises only one IL-21 mutein (i.e., the asymmetric multispecific antibody comprises an IL-21 mutein monomer) and the heavy chain contains the V131 charge pair mutations, the IL-21 mutein may be attached to the heavy chain containing the K360E, K370E, K392E, and K409D mutations (e.g., the IL-21 mutein is attached to a heavy chain comprising SEQ ID NO: 478, 480, or 482), or the heavy chain containing the E357K and D399K mutations (e.g., the IL-21 mutein is attached to a heavy chain comprising SEQ ID NO: 479, 481, or 483). In a specific embodiment, the IL-21 mutein is attached to the heavy chain containing the E357K and D399K mutations.

In exemplary aspects, one or both heavy chains of the asymmetric multispecific antibody comprises a constant region comprising a peptide linker (e.g., SEQ ID NO: 262), SEFL or SEFL2-2 mutations, HLE modifications, a clipped C-terminal Lys, charge pair mutations, or any combination thereof. In exemplary instances, one or both heavy chains of the asymmetric multispecific antibody comprises a constant region comprising an amino acid sequence of any one of SEQ ID NOs: 265, 266, 267, 282, 284, 285, 286, 287, 288, 289, 290, 291, 292, 293, 294, 295, 296, 297, 298, 299, 300, 301, 302, 303, 304, 305, 306, 307, 308, 309, 310, 311, and 472-495.

In some embodiments the asymmetric multispecific antibody comprises a single IL-21 mutein attached to an antibody that binds to a protein of the immune checkpoint pathway selected from the group consisting of: CTLA-4, PD-1, PD-L1, PD-L2, B7-H3, B7-H4, CEACAM-1, TIGIT, LAG3, CD112, CD112R, CD96, TEV13, BTLA, or co-stimulatory receptor: ICOS, OX40, 41BB, CD27, GITR.

In a related embodiment the antibody is an anti-PD-1 antibody. Suitable PD-1 antibodies are known in the art and include but are not limited to nivolumab (BMS-936558), pembrolizumab (MK-3475), BMS 936558, BMS- 936559, TSR-042 (Tesaro), ePDR001 (Novartis), and pidilizumab (CT-011), as well as any of the anti-PD-1 antibodies disclosed in International Patent Publication No. WO 2019/140196, which is incorporated herein by reference. In various instances, the anti-PD-1 antibody comprises (a) a heavy chain (HC) complementarity-determining region (CDR) 1 amino acid sequence selected from the group consisting of: SEQ ID NOs: 312, 322, 332, 342, 352, 362, 372, and 382; (b) an HC CDR2 amino acid sequence selected from the group consisting of: SEQ ID NOs: 313, 323, 333, 343, 353, 363, 373, and 383; (c) an HC CDR3 amino acid sequence selected from the group consisting of: SEQ ID NOs: 314, 324, 334, 344, 354, 364, 374, and 384; (d) a light chain (LC) CDR1 amino acid sequence selected from the group consisting of: SEQ ID NOs: 315, 325, 335, 345, 355, 365, 375, and 385; (e) an LC CDR2 amino acid sequence selected from the group consisting of: SEQ ID NOs: 316, 326, 336, 346, 356, 366, 376, and 386; (f) an LC CDR3 amino acid sequence selected from the group consisting of: SEQ ID NOs: 317, 327, 337, 347, 357, 367, 377, and 387; or (g) a combination of any two, three, four, five, or six of (a)-(f). In some embodiments, the anti-PD-1 antibody protein product comprises a HC CDR1, HC CDR2, HC CDR3, LC CDR1, LC CDR2, LC CDR3, according to Table 2.

TABLE 2
Heavy chain (HC) and light chain (LC) CDRs

| | 20A2 | 20C1 | 22D4 | 20C1.006 | 20C1.009 | 20A2.003 | 22D4.006 | 22D4.017 |
|---|---|---|---|---|---|---|---|---|
| HC CDR1 | 312 | 322 | 332 | 342 | 352 | 362 | 372 | 382 |
| HC CDR2 | 313 | 323 | 333 | 343 | 353 | 363 | 373 | 383 |
| HC CDR3 | 314 | 324 | 334 | 344 | 354 | 364 | 374 | 384 |
| LC CDR1 | 315 | 325 | 335 | 345 | 355 | 365 | 375 | 385 |
| LC CDR2 | 316 | 326 | 336 | 346 | 356 | 366 | 376 | 386 |
| LC CDR3 | 317 | 327 | 337 | 347 | 357 | 367 | 377 | 387 |

In another embodiment the anti-PD-1 antibody comprises two light chains, each comprising a LC CDR1, LC CDR2, and LC CDR3 comprising the amino acid sequence of SEQ ID NOs: 385, 386, and 387, respectively; and two heavy chains, each comprising a HC CDR1, HC CDR2, and HC CDR3 comprising the amino acid sequence of SEQ ID NOs: 382, 383, and 384, respectively. In one related embodiment each light chain variable region comprises the amino acid sequence of SEQ ID NO: 389 and each heavy chain variable region comprises the amino acid sequence of SEQ ID NO: 388. In one related embodiment each light chain comprises the amino acid sequence of SEQ ID NO: 391 and each heavy chain comprises the amino acid sequence of SEQ ID NO: 390.

In some embodiments the asymmetric multispecific antibody is an anti-PD1 antibody comprising an IL-21 mutein attached to the C-terminus of one of the two antibody heavy chains of the anti-PD-1 antibody. In some embodiments a single IL-21 mutein is attached to the Fc of one of the two antibody heavy chains of the anti-PD-1 antibody. In some embodiments the single IL-21 mutein is directly attached to the Fc of the anti-PD-1 antibody, e.g., the single IL-21 mutein is attached to the Fc of the anti-PD-1 antibody without a linker. In some embodiments the single IL-21 mutein is attached to the Fc of the anti-PD-1 antibody with a linker (e.g., a peptide linker comprising SEQ ID NO: 262), e.g., a peptide linker connects the single IL-21 mutein to the Fc of the anti-PD-1 antibody.

In some embodiments the asymmetric multispecific antibody comprises two light chains, each comprising the amino acid sequence of SEQ ID NO: 391; a single heavy chain attached to an IL-21 mutein comprising the amino acid sequence of any one of SEQ ID NOs: 501-506; and a heavy chain comprising the amino acid sequence of any one of SEQ ID NOs: 556-558. In some embodiments the asymmetric multispecific antibody comprises two light chains, each comprising the amino acid sequence of SEQ ID NO: 391; one heavy chain comprising the amino acid sequence of SEQ ID NO: 556; and one heavy chain attached to an IL-21 mutein comprising the amino acid sequence of SEQ ID NO: 501 In some embodiments the asymmetric multispecific antibody comprises two light chains, each comprising the amino acid sequence of SEQ ID NO: 391; one heavy chain comprising the amino acid sequence of SEQ ID NO: 557; and one heavy chain attached to an IL-21 mutein comprising the amino acid sequence of SEQ ID NO: 502. In some embodiments the asymmetric multispecific antibody comprises two light chains, each comprising the amino acid sequence of SEQ ID NO: 391; one heavy chain comprising the amino acid sequence of SEQ ID NO: 558; and one heavy chain attached to an IL-21 mutein comprising the amino acid sequence of SEQ ID NO: 503. In some embodiments the asymmetric multispecific antibody comprises two light chains, each comprising the amino acid sequence of SEQ ID NO: 391; one heavy chain comprising the amino acid sequence of SEQ ID NO: 556; and one heavy chain attached to an IL-21 mutein comprising the amino acid sequence of SEQ ID NO: 504. In some embodiments the asymmetric multispecific antibody comprises two light chains, each comprising the amino acid sequence of SEQ ID NO: 391; one heavy chain comprising the amino acid sequence of SEQ ID NO: 557; and one heavy chain attached to an IL-21 mutein comprising the amino acid sequence of SEQ ID NO: 505. In some embodiments the asymmetric multispecific antibody comprises two light chains, each comprising the amino acid sequence of SEQ ID NO: 391; one heavy chain comprising the amino acid sequence of SEQ ID NO: 558; and one heavy chain attached to an IL-21 mutein comprising the amino acid sequence of SEQ ID NO: 506.

In another embodiment the anti-PD-1 antibody comprises two light chains, each comprising a LC CDR1, LC CDR2, and LC CDR3 comprising the amino acid sequence of SEQ ID NOs: 365, 366, and 367, respectively; and two heavy chains, each comprising a HC CDR1, HC CDR2, and HC CDR3 comprising the amino acid sequence of SEQ ID NOs:362, 363, and 364, respectively. In one related embodiment each light chain variable region comprises the amino acid sequence of SEQ ID NO: 369 and each heavy chain variable region comprises the amino acid sequence of SEQ ID NO: 368. In one related embodiment each light chain comprises the amino acid sequence of SEQ ID NO: 371 and each heavy chain comprises the amino acid sequence of SEQ ID NO: 370.

In some embodiments the asymmetric multispecific antibody comprises two light chains, each comprising the amino acid sequence of SEQ ID NO: 371; a single heavy chain attached to an IL-21 mutein comprising the amino acid sequence of any one of SEQ ID NOs: 513-518; and a heavy chain comprising the amino acid sequence of any one of SEQ ID NOs: 559-561. In some embodiments the asymmetric multispecific antibody comprises two light chains, each comprising the amino acid sequence of SEQ ID NO 371; one heavy chain comprising the amino acid sequence of SEQ ID NO: 559; and one heavy chain attached to an IL-21 mutein comprising the amino acid sequence of SEQ ID NO: 513. In some embodiments the asymmetric multispecific antibody comprises two light chains, each comprising the amino acid sequence of SEQ ID NO: 371; one heavy chain comprising the amino acid sequence of SEQ ID NO: 560; and one heavy chain attached to an IL-21 mutein comprising the amino acid sequence of SEQ ID NO: 514. In some embodiments the asymmetric multispecific antibody comprises two light chains, each comprising the amino acid sequence of SEQ ID NO: 371; one heavy chain comprising the amino acid sequence of SEQ ID NO: 561; and one heavy chain attached to an IL-21 mutein comprising the amino acid sequence of SEQ ID NO: 515. In some embodiments the asymmetric multispecific antibody comprises two light chains, each comprising the amino acid sequence of SEQ ID NO: 371; one heavy chain comprising the amino acid sequence of SEQ ID NO: 559; and one heavy chain attached to an IL-21 mutein comprising the amino acid sequence of SEQ ID NO: 516. In some embodiments the asymmetric multispecific antibody comprises two light chains, each comprising the amino acid sequence of SEQ ID NO: 371; one heavy chain comprising the amino acid sequence of SEQ ID NO: 560; and one heavy chain attached to an IL-21 mutein comprising the amino acid sequence of SEQ ID NO: 517. In some embodiments the asymmetric multispecific antibody comprises two light chains, each comprising the amino acid sequence of SEQ ID NO: 371; one heavy chain comprising the amino acid sequence of SEQ ID NO: 561; and one heavy chain attached to an IL-21 mutein comprising the amino acid sequence of SEQ ID NO: 518.

The invention provides a method for modulating the product quality of a recombinant asymmetric multispecific antibody comprising a mutein comprising the steps: a) establishing at least two cell cultures each inoculated with the same cell line expressing the asymmetric multispecific antibody; b) culturing at least one cell culture at a first temperature regime that consists of a single temperature and at least one cell culture at a second temperature regime; c) comparing at least one product-related impurity in the cell culture at each temperature regime; and d) selecting the temperature regime that reduces the expression of at least one product-related impurity comprising an unpaired or mis-paired long heavy chain; wherein amount of at least one product-related impurity resulting from an imbalance in the ratio of long heavy chain to short heavy chain in the harvested cell culture is decreased compared to the amount of the same product-related impurity in the harvest from a cell culture exposed to a temperature of 32° C. to 34° C. at some point during the culture.

The invention provides a method for modulating the product quality of an asymmetric multispecific anti-PD-1 antibody comprising an IL-21 mutein attached to the C-terminus of one of the two antibody heavy chains of the anti-PD-1 antibody expressed by a cell during cell culture comprising the steps: a) establishing a cell culture inoculated with a cell line expressing the antibody; culturing the cells at 36±1° C. for the duration of the culture; and harvesting the antibody; wherein amount of at least one product-related impurity resulting from an imbalance in the ratio of long heavy chain to short heavy chain in the harvested cell culture is decreased compared to the amount of the same product-related impurity in the harvest from a cell culture exposed to a temperature of 32° C. to 34° C. at some point during the culture.

The invention provides a method for establishing a cell line from a clonally derived culture expressing an asymmetric multispecific antibody comprising the steps: a) transforming cells with a gene encoding an asymmetric multispecific antibody; b) isolating at least one single stably transformed cell expressing the asymmetric multispecific antibody and establishing a clonally derived culture; c) establishing at least two cell cultures from one or more clonally derived cultures; d) culturing at least one cell culture from each clonally derived culture at a first temperature regime and culturing at least one cell culture from each clonally derived culture at a second temperature regime; e) culturing at least one cell culture at a first temperature regime that consists of a single temperature and at least one cell culture at a second temperature regime; f) comparing the molar ratio of long heavy chain to short heavy chain produced by the cells at each temperature regime; g) selecting at least one clonally derived culture that produced a balanced or lower molar ratio of long heavy chain to short heavy chain; and h) establishing a cell line from the clonally derived culture.

The invention provides a method for producing an isolated, purified, recombinant asymmetrical multispecific antibody, the method comprising the steps: a) isolating at least one single stably transfected cell expressing the asymmetric multispecific antibody and establishing a clonally derived culture; b) establishing at least two cell cultures from one or more of the clonally derived cultures; c) culturing at least one cell culture from each clonally derived culture at a first temperature regime comprising a single temperature and culturing at least one cell culture from each clonally derived culture at a second temperature regime; d) comparing the amount of at least one product-related impurity comprising an unpaired or mis-paired long heavy chain produced by the cells cultured at each temperature regime; e) selecting the cell culture that modulated production of the product related impurity; f) establishing a cell line expressing the asymmetrical multispecific antibody from the selected clonally derived culture; g) inoculating a bioreactor with the cell line expressing the asymmetrical multispecific antibody; h) culturing the host cells to express the asymmetrical multispecific antibody at the temperature regime that modulated the production of the product related impurity; i) harvesting the recombinant asymmetrical multispecific antibody from the cell culture; j) processing the recombinant asymmetrical multispecific antibody through one or more chromatography unit operations, and k) obtaining an isolated, purified, recombinant asymmetrical multispecific antibody. In one embodiment is provided an isolated, purified, recombinant asymmetrical multispecific antibody made according the method above. In one embodiment is provided a pharmaceutical composition comprising the isolated, purified, recombinant asymmetrical multispecific antibody made according to the method above.

Also provided herein is a method for controlling cell growth in a nanofluidic chamber of a nanofluidic chip through one or more temperature shifts to extend cell culture duration, allow for collecting additional measurements of protein secretion levels and minimizing the risks of cell cross contamination during export procedure executed under reduced temperature conditions. Single cells expressing a recombinant protein, such as a monoclonal antibody, multispecific antibody, asymmetric protein, and the like, are deposited on a nanofluidic chip and subjected to static temperature culture where a constant temperature is maintained for the duration of the culture, or a biphasic culture where after an initial incubation at a first temperature condition, a temperature shift to one or more lower temperatures is applied. The temperature reduction led to growth inhibition, preventing cell overgrowth, reducing cell-cross contamination risks, and enabled long-term culture. while altering recombinant protein production profiles.

The method makes use of nanofluidic cell culture for screening thousands of clonally derived cell lines that are expanded and evaluated for secretion of desired therapeutic biologics. In one embodiment a nanofluidic cell culture system is a Beacon® Optofludic system (Berkeley Lights, Inc., Emeryville, CA), a fully integrated nanofluidic cell culture system that makes use of nanofluidic chips that allow for the isolation of thousands of clonal cell lines. The Beacon® Optofludic platform is suitable for cell line development workflow and enables assessment of growth and desired secretory profiles at the single cell or a few cell levels. Cells are loaded into nanofluidic chambers of a nanofluidic chip and can be simultaneously cultured and assayed for recombinant protein secretion. Candidate clones are selected based on desired growth and secretory profiles and exported off the chip and subjected to typical scale-up workflows. Additional single-cell sorting and analytical platforms are also contemplated for use with the methods of the present disclosure. In one embodiment, a lithographic-based microarrays or nanowell-assisted cell patterning platforms (Love et al., Nat. Biotechnol., 24(6) 703 (2006); and Ozkumur et al., Materials Views, 11(36). 4643-4650 (2015) is contemplated.

As used herein. “nanofluidic chamber of a nanofluidic chip” refers to a portion or section of a nanofluidic device that is capable of isolating a single cell. By way of example, a nanofluidic chamber can be a NanoPen™ chamber associated with Berkeley Light's Beacon® technology platform as described herein. In some embodiments, the nanofluidic chip or device comprises hundreds or thousands of individual chambers each capable of isolating a single cell. In some embodiments, the nanofluidic device or chip comprises 1758 chambers, 3,500 chambers, 11,000 chambers, 14,000 chamber, or 20,000 chambers. Such nanofluidic chips are known in the art and available commercially, BLI OptoSelect™ Chip, BLI OptoSelect™ Chip 1750b, BLI OptoSelect™ Chip 3500, BLI OptoSelect™ Chip 11k, BLI OptoSelect™ Chip 14K, BLI OptoSelect™ Chip 20k.

Methods for producing biomolecules selected using the method are also contemplated by the present disclosure. For example, in one embodiment, a biomolecule grown under the conditions of the method are exported to multi-well plates, such as 96-, 12-, or 24-well plates. The effects of static and shifted temperature on the recombinant protein are analyzed and used to aid early clone selection. Cell lines that secrete a recombinant protein having desired properties and product quality attributes are isolated and transferred to a vessel for culturing under conditions that allow production of the recombinant protein in vessels and under conditions described herein.

The invention provides a method for controlling the growth of cells in a nanofluidic chamber of a nanofluidic chip comprising the steps of: (a) isolating a single cell into a nanofluidic chamber of a nanofluidic chip, wherein said cell comprises an expression construct capable of expressing a recombinant protein; (b) culturing the cell at a first temperature; (c) at a predetermined point, culturing the cells at a second temperature; and (d) exporting the cells out of the nanofluidic chamber and into a culture vessel. In one embodiment the number of cells per chamber at export is lower compared to a single cell derived cell line cultured under similar conditions at a constant temperature for the duration of the culture.

The invention also provides a method for minimizing cell cross contamination during export of cells grown in a nanofluidic chamber of a nanofluidic chip comprising the steps of: (a) isolating a single cell into a nanofluidic chamber of a nanofluidic chip, wherein said cell comprises an expression construct capable of expressing a recombinant protein; (b) culturing the cell at a first temperature; (c) at a predetermined point. culturing the cells at a second temperature; and (d) exporting the cells out of the nanofluidic chamber and into a culture vessel at the lower temperature setting applied during the export procedure. The number of cells per chamber at export is lower compared to a single cell cultured under similar conditions a constant temperature for the duration of the culture.

The invention further provides a method to improve clone selection for cells grown in a nanofluidic chamber of a nanofluidic chip comprising the steps of: (a) isolating a single cell into a nanofluidic chamber of a nanofluidic chip, wherein said cell comprises an expression construct capable of expressing a recombinant protein; (b) culturing the cell at a first temperature; (c) lowering the temperature of the culture to a second temperature no earlier than day 3 of the culture; and (d) exporting the cells out of the nanofluidic chamber at least one to four days after the temperature shift.

In one embodiment exporting the cells out of the nanofluidic chamber is at least 2-4 days after the temperature shift. In one embodiment exporting the cells out of the nanofluidic chamber is at least 3-4 days after the temperature shift. In one embodiment exporting the cells out of the nanofluidic chamber is at least 2-3 days after the temperature shift. In one embodiment exporting the cells out of the nanofluidic chamber is at least 1-2 days after the temperature shift. In one embodiment, exporting the cells out of the nanofluidic chamber is at least 1, 2, 3, or 4 days after the temperature shift. In one embodiment, exporting the cells out of the nanofluidic chamber is at least 1 day after the temperature shift. In one embodiment, exporting the cells out of the nanofluidic chamber is at least 2 days after the temperature shift. In one embodiment, exporting the cells out of the nanofluidic chamber is at least 3 days after the temperature shift. In one embodiment, exporting the cells out of the nanofluidic chamber is at least 4 days after the temperature shift.

In one embodiment the first temperature is selected from 35° C. to 37° C. In one embodiment the first temperature is 35° C., 36° C., or 37° C. In one embodiment the first temperature is 35° C. In one embodiment the first temperature is 36° C. In one embodiment the first temperature is 37° C.

In one embodiment the second temperature is selected from 28° C. to 34° C. In one embodiment the second temperature is selected from 28° C., 29° C., 30° C., 31° C., 32° C., 33° C., or 34° C.±0.5° C. In one embodiment the second temperature is selected from 28° C. In one embodiment the second temperature is selected from 29° C. In one embodiment the second temperature is selected from 30° C. In one embodiment the second temperature is selected from 31° C. In one embodiment the second temperature is selected from 32° C. In one embodiment the second temperature is selected from 33° C. In one embodiment the second temperature is selected from 34° C.

In one embodiment the second temperature is about 1° C. to about 9° C. lower than the first temperature. In one embodiment the first temperature is 36° C. and the second temperature is 32° C. to 32.5° C.

In one embodiment the predetermined point is on day 3 to day 5 of the culture. In one embodiment the predetermined point is on day 3 to day 4 of the culture. In one embodiment the predetermined point is on day 4 to day 5 of the culture. In one embodiment the predetermined point is on day 3, day 4 or day 5 of the culture. In one embodiment the predetermined point is on day 3 of the culture. In one embodiment the predetermined point is on day 4 of the culture. In one embodiment the predetermined point is on day 5 of the culture.

In one embodiment export is on day 6 to day 8 of the culture. In one embodiment export is on day 6 to day 7 of the culture. In one embodiment export is on day 7 to day 8 of the culture. In one embodiment export is on day 6, day 7 or day 8 of the culture. In one embodiment export is on day 6 of the culture. In one embodiment export is on day 7 of the culture. In one embodiment export is on day 8 of the culture.

In one embodiment the nanofluidic chip comprises 1758 chambers, 3,500 chambers, 11,000 chambers, 14,000 chamber, or 20,000 chambers.

In one embodiment the culture vessel is a multi-well plate. In one embodiment the protein secretion profiles before and after the temperature shift are compared. In one embodiment the nanofluidic chip comprises 1758 chambers.

By “culture” or “culturing” is meant the growth and propagation of cells outside of a multicellular organism or tissue. Suitable culture conditions for mammalian cells are known in the art. Cell culture media and tissue culture media are used interchangeably to refer to media suitable for growth of a host cell during in vitro cell culture. Typically, cell culture media contains a buffer, salts, energy source, amino acids, vitamins and trace essential elements. Any media capable of supporting growth of the appropriate host cell in culture can be used. Cell culture media, which may be further supplemented with other components to maximize cell growth, cell viability, and/or recombinant protein production in a particular cultured host cell, are commercially available and include RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series, among others, which can be obtained from the American Type Culture Collection or SAFC Biosciences, STEMCELL Technologies Inc as well as other vendors. Cell culture media can be serum-free, protein-free, growth factor-free, and/or peptone-free media. Cell culture may also be enriched by the addition of nutrients and used at greater than its usual, recommended concentrations.

Various media formulations may be used at all stages of cell culture, from clone selection and cell line development and through large scale production cell culture. Different media formulations can be used during selection, to facilitate transition from one stage (e.g., the growth stage or phase) to another (e.g., the production stage or phase), and/or to optimize conditions during cell culture (e.g. concentrated media provided during perfusion culture, feed media during fed batch culture). A selection media can be formulated to apply selective pressure to newly transformed cells. A growth medium formulation can be used to promote cell growth and minimize protein expression. A production medium formulation can be used to promote production of the protein of interest and maintenance of the cells, with a minimal of new cell growth). A feed media, typically a media containing more concentrated components such as nutrients and amino acids, which are consumed during the course of the production phase of the cell culture may be used to supplement and maintain an active culture, particularly a culture operated in fed batch, semi-perfusion, or perfusion mode. Such a concentrated feed medium can contain most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal amount.

Cell cultures can be operated in a batch, fed batch, continuous, semi-continuous, perfusion mode or any combination therein.

Temperature shifts are commonly used in Chinese hamster ovary (CHO) cell cultivation to influence the performance of the culture and recombinant expression of proteins. CHO cells are typically grown at physiological temperature, around 36° C.-37° C. A temperature shift to a hypothermic condition around 30° C.-34° C. is used to control such aspects of the culture as cell growth, improve recombinant protein production, yield, and/or maintain cell viability, and the like.

In addition, chemical inducers of protein production, such as, for example, caffeine, butyrate, and hexamethylene bisacetamide (HMBA), may be added to the cell culture. If there is a temperature shift, the inducers may be added at the same time as, before, and/or after a temperature shift. If inducers are added after a temperature shift, they can be added from one hour to five days after the temperature shift, optionally from one to two days after the temperature shift. pH may also be shifted during culture, either independently or in combination with other methods.

Host cells may be cultured in suspension or in an adherent form, attached to a solid substrate. Cells may be cultured in multi-well plates, such as 96, 24, or 6 well and deep well plates. Cell cultures can be established in fluidized bed bioreactors, gas permeable culture bags, gas permeable bioreactors, hollow fiber bioreactors, roller bottles, spin tubes, shake flasks, or stirred tank bioreactors, with or without microcarriers. Mammalian cells, such as CHO cells, may be cultured in bioreactors at a smaller scale of less than 100 ml to less than 1000 mls. Alternatively, larger scale bioreactors that contain 1000 mls to over 20,000 liters of media can be used. Large scale cell cultures, such as for clinical and/or commercial scale biomanufacturing of protein therapeutics, may be maintained for weeks and even months, while the cells produce the desired protein(s).

The term “unit operation” refers to a functional step that is performed as part of the process of purifying a recombinant protein of interest. For example, a unit operation can include steps in operations such as, but not limited to, harvest, capture, purification, polish, viral inactivation, virus filtration, concentration and/or formulation the recombinant protein of interest. Unit operations can be designed to achieve a single objective or multiple objectives, such as a combination of capture and virus inactivation steps. Unit operations can also include holding or storing steps between processing steps.

The recombinant protein is harvested from the cell culture media. Methods for harvesting protein from suspension cells are known in the art and include, but are not limited to, acid precipitation, accelerated sedimentation such as flocculation, separation using gravity, centrifugation, acoustic wave separation, filtration, including membrane filtration using ultrafilters, microfilters, tangential flow filters, alternative tangential flow filters, depth filters, and alluvial filters. Recombinant proteins expressed by prokaryotes are retrieved from inclusion bodies in the cytoplasm by processes incorporating redox folding processes known in the art.

The harvested protein can then be purified, or partially purified, away from any impurities, such as remaining cell culture media, cell extracts, undesired components, host cell proteins, improperly expressed proteins and the like, using one or more unit operations. By “purifying” is meant increasing the degree of purity of the asymmetric multispecific antibody in the composition by removing (partially or completely) at least one impurity or contaminant from the composition. Recovery and purification of multispecific antibody is accomplished by the downstream unit operations, in particular, those operations involving chromatography, resulting in a more “homogeneous” asymmetric multispecific antibody composition that meets yield and product quality targets.

A capture unit operation may include capture chromatography that makes use of resins and/or membranes containing agents that will bind and/or interact with at least one desired protein, impurity or contaminant. Examples of capture chromatography include affinity chromatography, size exclusion chromatography, ion exchange chromatography, hydrophobic interaction chromatography (HIC), immobilized metal affinity chromatography (IMAC), and the like. Such materials are known in the art and are commercially available. Affinity chromatography is commonly used in biomanufacturing processes as an initial capture step to isolate and concentrate recombinant proteins of interest having an Fc component. Examples of such affinity chromatography materials include those that make use of Staphylococcus proteins such as Protein A, Protein G, Protein A/G, and Protein L; substrate-binding capture mechanisms; antibody- or antibody fragment-binding capture mechanisms; aptamer-binding capture mechanisms; cofactor-binding capture mechanisms; and the like. Immobilized metal affinity chromatography can be used to capture proteins that have or have been engineered to have affinity for metal ions. Affinity chromatography material is available commercially from a number of vendors. For example, MABSELECT™ SURE Protein A, Protein A Sepharose FAST FLOW™ (Cytiva, Marborough, MA), PROSEP-A™ (Merck Millipore, U.K), TOYOPEARL™ 650M Protein A (TosoHass Co., Philadelphia, PA).

Intermediate and/or polishing unit operations make use of various chromatography methods for the continued purification of the protein of interest and clearance of contaminants and impurities such as DNA, host cell proteins; removal of product-specific impurities, variant products and aggregates, virus adsorption, and the like. These chromatography unit operations makes use of resins and/or membranes containing agents that can be used in either a flow-through mode where the protein of interest is contained in the eluent and the contaminants and impurities are bound to the chromatography medium; frontal or overloaded chromatography mode where a solution containing the protein of interest is loaded onto a column until adsorption sites on arc occupied and the species with the least affinity for the stationary phase (the protein of interest) starts to elute; bind and elute mode, where the protein of interest is bound to the chromatography medium and eluted after the contaminants and impurities have flowed through or been washed off the chromatography medium, or any other method. Examples of such chromatography methods include ion exchange chromatography (IEX), such as anion exchange chromatography (AEX) and cation exchange chromatography (CEX); hydrophobic interaction chromatography (HIC); mixed modal or multimodal chromatography (MM), hydroxyapatite chromatography (HA); reverse phase chromatography and gel filtration, among others.

Multiple chromatography unit operations, typically one, two, or three, each performing a different function, are combined depending on the requirements of the manufacturing process. Ion exchange chromatography, based on electrostatic interactions between charged surfaces, separates proteins of interest from impurities based on differential absorption and desorption. Cation exchange chromatography refers to chromatography performed on a solid phase medium that is negatively charged and has free cations for exchange with cations in an aqueous solution passed over or through the solid phase. The charge may be provided by attaching one or more charged ligands to the solid phase, e.g. by covalent linking. Alternatively. or in addition, the charge may be an inherent property of the solid phase (e.g. as is the case for silica, which has an overall negative charge). Cation exchange chromatography is typically run in bind and elute mode, the high pI of many proteins of interest enable binding to the chromatography material. Cation exchange chromatography may also be run in flow through mode. CEX chromatography is typically used to remove high molecular weight (HMW) contaminants, process related impurity, and/or viral clearance. Commercially available cation exchange mediums are available and include but are not limited to sulphopropyl (SP) immobilized on agarose (e.g. SP-SEPHAROSE FAST FLOW™, SP-SEPHAROSE FAST FLOW XL™ or SP-SEPHAROSE HIGH PERFORMANCE™, CAPTO S™, CAPTO SP ImpRes™, CAPTO S ImpAct™ (Cytiva), FRACTOGEL-SO3™, FRACTOGEL-SE HICAP™, and FRACTOPREP™ (EMD Merck, Darmstadt, Germany), TOYOPEARL® XS, TOYOPEARL® HS (Tosh Bioscience, King of Prussia, PA), UNOsphere™ (BioRad, Hercules, CA), S Ceramic Hyper D™ F (Pall, Port Washington, NY), POROS™ (ThermoFisher, Waltham, MA).

Anion exchange chromatography refers to chromatography performed on a solid phase medium that is positively charged and has free anions for exchange with anions in an aqueous solution passed over or through the solid phase. Anion exchange chromatography is typically run in flow through mode. Due to the high pI of many proteins of interest they do not to bind to the AEX chromatography material. AEX chromatography is used, for example, for viral clearance and impurity removal. Commercially available anion exchange mediums are available and include, but are not limited to, sulphopropyl (SP) immobilized on agarose (e.g. Source 15 Q, Capto™ Q, Q-SEPHAROSE FAST FLOW™ (Cytiva), FRACTOGEL EDM TMAE™, FRACTOGEL EDM DEAE™ (EMD Merck), TOYOPEARL Super Q® (Tosh Bioscience), POROS HQ™, POROS XQ™, (ThermoFisher).

Mixed-mode or multi-mode chromatography (MMC) refers to chromatography performed on a solid phase medium that makes use of a combination of interaction mechanisms, such as ion exchange (CEX or AEX) and hydrophobic interaction, and others. Commercially available multi-modal chromatography media are available and include Capto™ Adhere (Cytiva).

Hydrophobic interaction chromatography refers to chromatography performed on a solid phase medium that makes use of the interaction between hydrophobic ligands and hydrophobic residues on the surface of a protein of interest. Commercially available hydrophobic interaction chromatography media includes but are not limited to Phenyl Sepharose™ (Cytiva), Tosoh hexyl (Tosoh Bioscience), and Capto™ phenyl (Cytiva).

Hydroxyapatite chromatography refers to chromatography performed on a solid phase medium that makes use of positively charged calcium and negatively charged phosphate and depending on the pI of the protein and the pH of the buffer, can act as a cation or anion.

Unit operations comprising inactivating, reducing and/or eliminating viral contaminants may include processes that manipulate the environment and/or filtration. Various methods can be employed for virus inactivation and include heat inactivation/pasteurization, UV and gamma ray irradiation, use of high intensity broad spectrum white light, addition of chemical inactivating agents, surfactants, and solvent/detergent treatments. Surfactants, such as detergents, solubilize membranes and therefore can be very effective in specifically inactivating enveloped viruses. One method for achieving virus inactivation is incubation at low pH (e.g., pH<4). Low pH virus inactivation can be followed with a neutralization unit operation that readjusts the viral inactivated solution to a pH more compatible with the requirements of the following unit operations. It may also be followed by filtration, such as depth filtration, to remove any resulting turbidity or precipitation. Viral filtration can be performed using micro- or nano-filters, such as those available from Asahi Kasei (Plavona®) and EDM Millipore (VPro®).

Unit operations may also comprise product concentration and buffer exchange of the protein of interest into a desired formulation buffer for bulk storage of the drug substance can be accomplished using known methods for ultrafiltration and diafiltration (UF/DF). Unit operations related to drug product fill/finish can follow.

Critical attributes and performance parameters of the purified asymmetric multispecific antibody can be measured to better inform decisions regarding performance of each step during manufacture. These critical attributes and parameters can be monitored real-time, near real-time, and/or after the fact. Key critical parameters such as media components that are consumed (such as glucose), levels of metabolic by-products (such as lactate and ammonia) that accumulate, as well as those related to cell maintenance and survival, such as dissolved oxygen content can be measured during cell culture. Critical attributes such as specific productivity, viable cell density, pH, osmolality, appearance, color, aggregation, percent yield and titer may be monitored during appropriated stages in the manufacturing process. Monitoring and measurements can be done using known techniques and commercially available equipment.

The pharmaceutical compositions (solutions, suspensions or the like), may include one or more of the following: buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives; sterile diluents such as water for injection, saline solution, preferably physiological saline, Ringer's solution, isotonic sodium chloride, fixed oils such as synthetic mono- or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

While the terminology used in this application is standard within the art, definitions of certain terms are provided herein to assure clarity and definiteness to the meaning of the claims. Units, prefixes, and symbols may be denoted in their SI accepted form. Numeric ranges recited herein are inclusive of the numbers defining the range and include and are supportive of each integer within the defined range. The methods and techniques described herein are generally performed according to conventional methods well known in the art and as described in various general and more specific references that are cited and discussed throughout the present specification unless otherwise indicated. All documents, or portions of documents, cited in this application, including but not limited to patents, patent applications, articles, books, and treatises, are hereby expressly incorporated by reference.

The present invention is not to be limited in scope by the specific embodiments described herein that are intended as single illustrations of individual aspects of the invention, and functionally equivalent methods and components are within the scope of the invention. What is described in an embodiment of the invention can be combined with other embodiments of the invention. Indeed, various modifications of the invention, in addition to those shown and described herein will become apparent to those skilled in the art from the foregoing description and accompanying drawings. Such modifications are intended to fall within the scope of the appended claims.

The following examples, including the experiments conducted and the results achieved, are provided for illustrative purposes only and are not to be construed as limiting the scope of the appended claims.

### EXAMPLES

### Example 1

CHO cells were transfected with a plasmid DNA encoding a bifunctional fusion protein comprising a conjugate of an IL-21 mutein linked to the C-terminus of one of the two antibody heavy chains of an anti-PD-1 antibody. The IL-21-anti-PD-1 conjugate comprised a “short heavy chain” (an anti-PD1 heavy chain) and a “long heavy chain” (an anti-PD 1 heavy chain linked to the IL-21 mutein). The weight, length and amino acid composition differed between the two heavy chains. Following transfection, pool populations stably expressing the conjugate were generated through repeated passaging in a selective growth medium. When the pools reached above 90% viability as determined by Trypan Blue Exclusion and maintained consistent doubling times, single cell clones were isolated from the pools.

Single cell clones were expanded and cultured in 96 well plates (Corning, Corning, NY) which were maintained at 36° C., 5% CO₂, 85% relative humidity. The cells were maintained by passaging until they reached a desired cell viability density.

The clones were then inoculated at 8×10⁵ cells/ml into 24 well plates (Axygen, Union City, CA) with a 3.5 ml working volume of serum free, chemically defined culture medium. The plates were maintained at 36° C., 5% CO₂, 85% relative humidity, shaken at 225 rpm for 10 days and fed on days 3, 6, and 8 at 7% post-inoculation volume. The clones were harvested on day 10. In-process samples were taken to monitor culture conditions. Viable cell density (VCD) and viability (%) were determined using a Vi-Cell XR Cell Viability Analyzer (Beckman Coulter, Indianapolis, IN). Glucose, lactate, and NH4+ concentrations were determined using a BioProfile Flex Analyzer (Nova Biomedical, Waltham, MA).

Samples from each culture were taken for titer determination and product quality analysis. Titers were measured by high performance liquid chromatography (HPLC) via affinity chromatography (Protein A, Waters, Milford, MA). Integrated viable cell density (IVCD) was calculated by a trapezoidal rule for VCD versus culture time. Cell specific productivity (qp) was calculated by titer divided by IVCD. Harvest material was used for product quality assessments. The top four clones were selected for bioreactor culture.

The top four clones (Clones 1-4) were inoculated at 10E5 cells/ml into 2×7 L bioreactors at a working volume of 4.4L serum free, chemically defined culture medium. The bioreactors were maintained at 36° C., 5% CO₂, 6.90 pH, 315 rpm. Perfusion was initiated on day 3 and used an alternating tangential flow filter system, (Repligen, Waltham, MA) with a 30,000 NMWC filter (GE Healthcare, Westborough, MA) at a rate of 0.5 reactor volumes/day on days 3-6; 0.75 reactor volumes/day for days 7-8; and 1.0 reactor volumes/day for days 9-15, except for Clone 2 which was stopped on day 14. Daily samples were taken to monitor culture conditions as described above for the 24 well plates (e.g., pH, osmolality, lactate/glucose, etc.).

When culture viable cell density (VCD) exceeded 300E5 cells/ml the temperature was shifted to 32.5° C. and the culture was maintained at that temperature until harvest. The clones were harvested from day 12 to day 15 using a 750,000 NMWC filter, GE Healthcare, except for Clone 2 which was harvested from day 12 to day 14. Culture conditions and harvested culture supernatant were monitored using the same methods as described above for the 24 well plates.

Unexpectedly, the samples exposed to the lower culture temperature (32.5° C.) showed an increase in the molar ratio of long heavy chain (LHC) to short heavy chain (SHC) for all clones tested.

A second experiment was performed to determine the cause of the increase in the molar ratio of long heavy chain to short heavy chain. A 10-day fed-batch culture was performed to study the response of Clone 4 cultured at a constant 36° C. and under conditions that incorporated a temperature shift during the culture (36° C. to 32.5° C.). Cells from Clone 4 were inoculated at 8×10⁵ cells/ml into two 24 deep-well plates (Axygen, Union City, CA) with a 3.5 ml working volume of serum free, chemically defined culture medium. The plates were maintained at 36° C., 5% CO₂, 85% relative humidity, shaken at 225 rpm. One plate was subjected to a temperature shift to 32.5° C., 5% CO₂, 225 rpm. Both cultures were fed on day 3, 6, and 8 at 7% of post-inoculation volume. The cells from both cultures were harvested on day 10. Analytical measurements were taken throughout the 10-day culture, as described above. The harvested material was then purified using Protein A affinity chromatography.

Cells from Clones 1-3 were also tested in a 15-day perfusion culture in 7 L bioreactors to study the protein response of the clones under perfusion conditions maintained a constant temperature of 36° C. throughout. Clone 4 was tested in a 15-day perfusion vulture in a 7 L bioreactor to study the protein response of the clone under perfusion conditions with a temperature shift (36° C.-32.5° C.).

The test clones and controls were inoculated at 10E5 cells/ml into 2×7 L bioreactors at a working volume of 4400 ml of serum free, chemically defined culture medium. The bioreactors were maintained at 36° C. 5% CO₂, 6.90 pH. 315 rpm. Perfusion was initiated on day 3 and used an alternating tangential flow filter system. (Repligen. Waltham. MA) with a 30,000 NMWC filter (GE Healthcare, Westborough, MA) at a rate of 0.5 reactor volumes/day on days 3-6; 0.75 reactor volumes/day for days 7-8; and 1.0 reactor volumes/day for days 9-15, except for Clone 2 which was stopped on day 14. Daily samples were taken to monitor culture conditions as described above. Clones 1 and 3 were harvested from day 12 to day 15 using a 750,000 NMWC filter, (GE Healthcare), except for Clone 2 which was harvested from day 12 to day 14. Monitoring culture conditions took place throughout. Analytical measurements were taken though out the cultures as described above. The harvested material was purified using Protein A affinity chromatography followed by cation exchange chromatography in bind and elute mode.

For both the fed-batch and perfusion samples, the molar ratio of long heavy chain to the short heavy chain was determined by reduced capillary electrophoresis sodium dodecyl sulfate (rCE-SDS). Mean Pre-Peak % was determined by non-reduced capillary electrophoresis-sodium dodecyl sulfate (nrCE-SDS). Mean LMW % was determined using size exclusion ultra high-performance liquid chromatography (SE-UHPLC). For both the fed-batch and perfusion samples, the molar ratio of long heavy chain to the short heavy chain was determined by reduced capillary electrophoresis-sodium dodecyl sulfate (rCE-SDS).

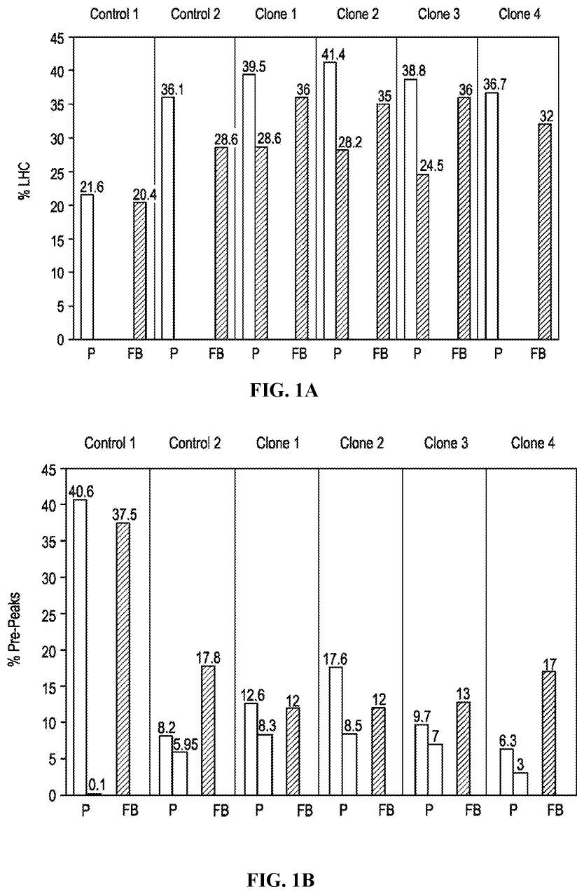

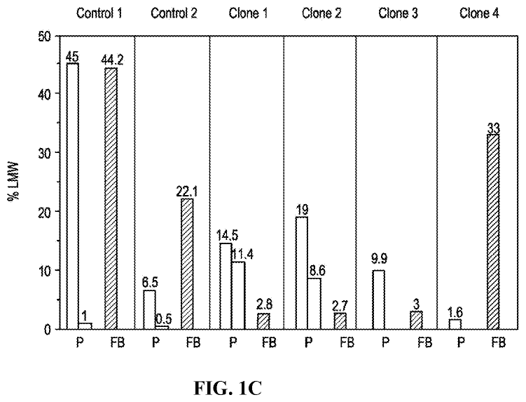

An increase in the % long heavy chain (% LHC) when cells expressing the IL-2-anti-PD-1 conjugate was subjected to perfusion culture (P) with a temperature shift to 32.5° C. (open bars), compared to fed batch culture (FB) at a constant 36° C. (striped bars), see FIG. 1A. The data confirm that the change in ratio was due to the temperature shift and not to the switch in culture methods. It was found that partial reduced species produced during the perfusion culture with a temperature shift from 36° C. to 32.5° C. were not possible to clear during downstream purification. A comparison of the Pre-peaks from the temperature shifted perfusion culture (P) subjected to purification by Protein A affinity chromatography (left open bar) and cation exchange chromatography (right open bar) and the constant temperature fed batch culture (FB) subjected to Protein A affinity chromatography (striped bar) is shown in FIG. 1B. The perfusion culture samples subjected to a temperature shift had a higher percentage of LMW than did the fed batch culture samples subjected to a constant temperature. See FIG. 1C. In particular, excess long heavy chain could not be separated by CEX purification. A chromatogram of the Pre-peak impurities characterized by nrCEX-SDS from the CEX pool is provided in FIG. 1D. Excess long heavy chain species from the perfusion samples could not be separated by CEX chromatography.

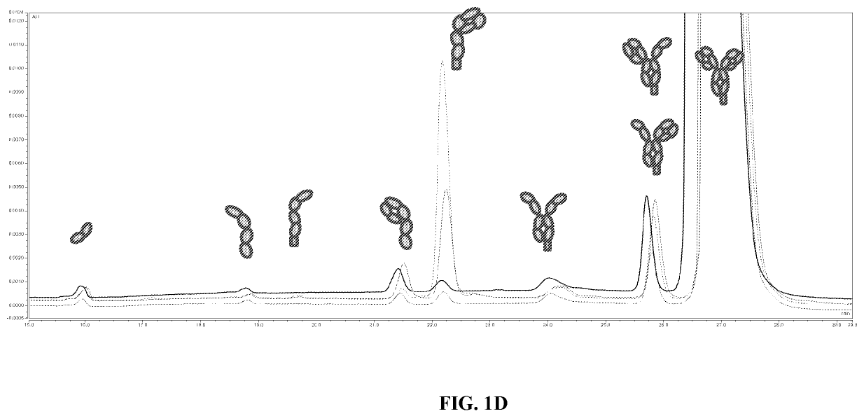

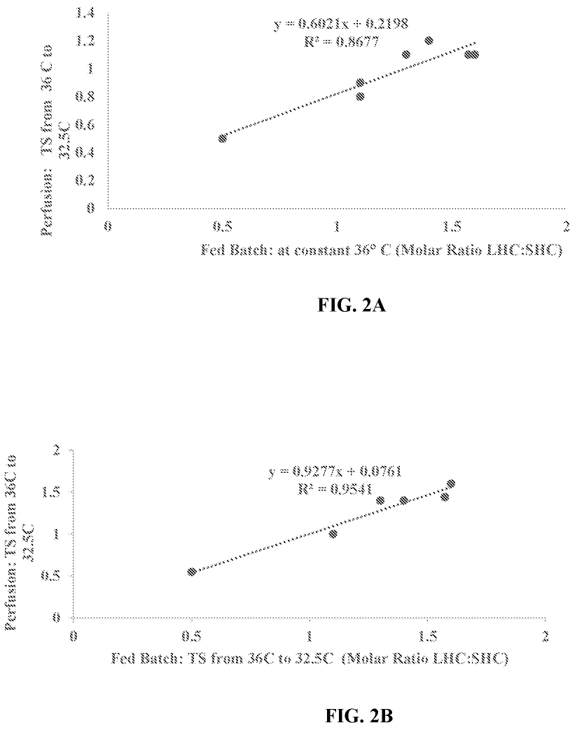

Comparing the molar ratio of the long heavy chain to the short heavy chain as determined by rCE showed that there was a better correlation between the fed batch samples and the perfusion samples if both were subjected to the same temperature regime. FIG. 2A shows fed batch culture samples grown at a constant 36° C. compared to perfusion samples grown with a temperature shift from 36° C. to 32.5° C. FIG. 2B shows fed batch culture samples compared to perfusion samples, both grown with a temperature shift from 36° C. to 32.5° C. The data points for both culture methods grown at a constant 36° C. were limited and not included in the comparison.

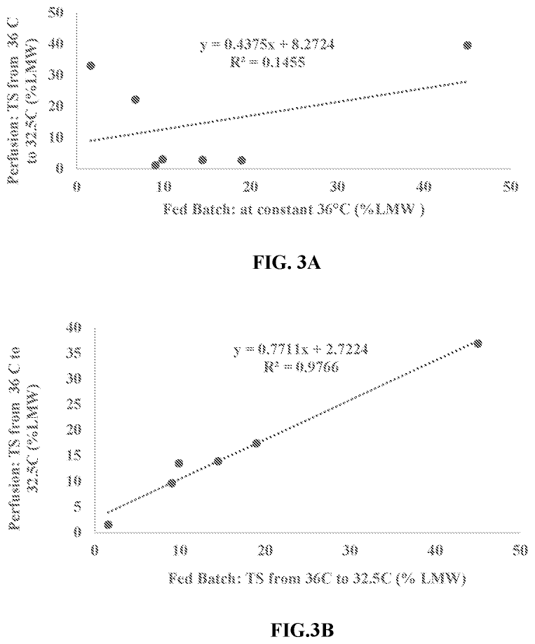

Comparing the % LMW determined by SEC showed that there was better correlation between the fed batch samples and the perfusion samples if they were subjected to the same temperature regime. FIG. 3A shows fed batch culture samples grown at a constant 36° C. compared to perfusion samples grown with a temperature shift from 36° C. to 32.5° C. FIG. 3B shows fed batch culture samples compared to perfusion samples, both grown with a temperature shift from 36° C. to 32.5° C. The data points for both culture methods grown at a constant 36° C. were limited and not included in the comparison.

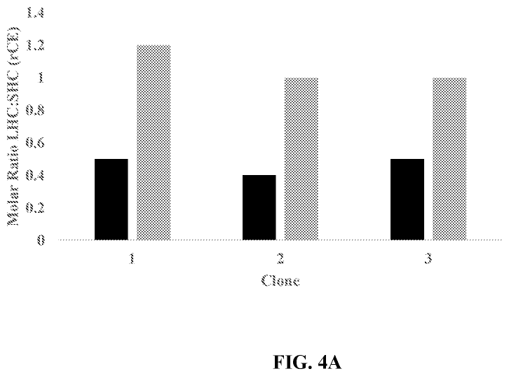

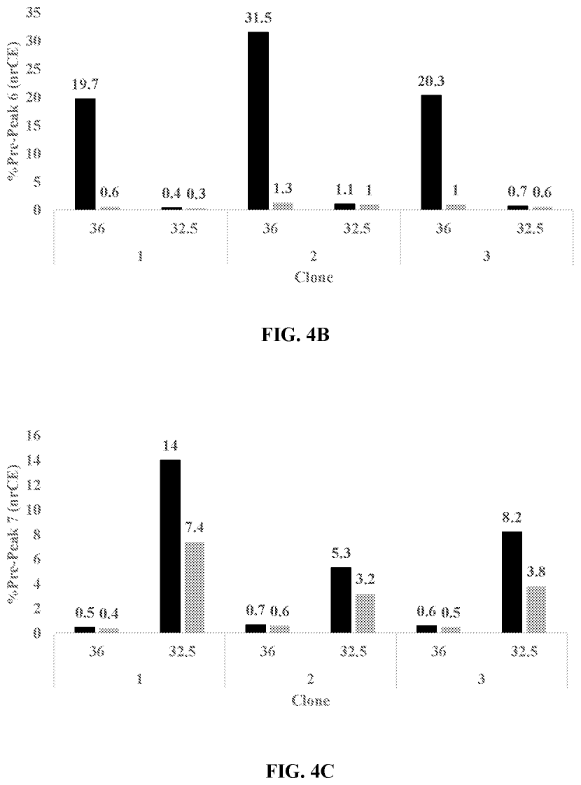

An unexpected shift in the Molar ratio of long heavy chain (LHC) to the short heavy chain (SHC) was seen when cultures were subjected to a temperature shift from 36° C. to 32.5° C. compared to cultures grown at a constant temperature of 36° C., (FIG. 4A Constant temperature of 36° C., Black Bars. Temperature shift from 36° C. to 32.5° C., Gray Bars). This imbalance impacted downstream purification (Protein A affinity chromatography and cation exchange chromatography) and product quality. Culture at the lower temperature (32.5° C.), resulted in a decrease of impurities formed by the short heavy chain (pre-peak 6) and increase of impurities formed by long heavy chain (pre-peak 7) as determined by nrCE. The short heavy chain impurities could be removed by Protein A and CEX chromatography purification (FIG. 4B) while the long heavy chain impurities were difficult to remove by the same purification systems (FIG. 4C). Protein A affinity chromatography (Black Bars) and cation exchange chromatography (Gray Bars).

This result illustrated that the cell culture temperature could impact the product quality by affecting the ratio of the two heavy chains, which impacted the impurities that were removed by the purification processes. Depending on the property of the impurities, different levels of purity could be achieved. In this example, higher purity was achieved at the higher cell culture temperature.

The results showed that there was better correlation between the product quality of the IL-2-anti-PD-1 conjugate when the clones were screened and selected at the same temperature regime expected to be encountered by the resulting cell lines. The molar ratio of the long heavy chain and a short heavy chain was found to be influenced by the temperature of the culture, with increased expression of the long heavy chain at lower temperatures. An increase in the ratio of long heavy chain to short heavy chain influenced the formation and type of product-related impurities. The impurities comprising the long heavy chain were found to be surprisingly more difficult to clear in downstream purification operations compared to impurities comprising the short heavy chain. This altered ratio of the long and short heavy chains had an impact on the purification, activity, product quality, as well as the robustness of the manufacturing process as a whole.

### Example 2

This experiment looked at the effect of temperature during clone selection and the impact on product quality at a transcriptional level for an asymmetric bispecific antibody, Bispecific A, comprising two heavy chains, a “long heavy chain” having the longer amino acid sequence of the pair and “short heavy chain” having the shorter amino acid sequence.

### Fed Batch Shaker Culture

A clone expressing a bispecific antibody (Bispecific A) was selected from a group of clones from the initial clonal cell screening, (Clone 1). A 10-day fed-batch clone screen was conducted in shake flasks to study the transcript and protein response during cell culture of the clone in a screening protocol comparing the performance of the clone when cultured at a constant temperature (36° C.) compared to culture with a temperature shift (36° C. to 32.5° C.).

A IL shake flask with a 250ml working volume of serum free, chemically defined culture medium was initiated. The flask was inoculated at 8×10⁵ cells/ml. The shake flask was maintained at 36° C., 5% CO₂, 160 rpm, for seven days and fed on days 3 and 6, at 7% of post-inoculation volume.

On day 7, the culture was split into two 250 ml shake flasks with a 60 ml working volume of serum free, chemically defined culture medium. One flask was maintained at 36° C., the culture temperature of the second flask was lowered to 32.5° C. Both flasks were cultured at 5% CO₂, agitation at 160 rpm, until day 10. The cultures were fed on day 8 at 7% of post-inoculation volume. Analytical measurements were taken throughout the 10-day culture, as described in the Example above. Integral viable cell density (IVCD) was measured at harvest using a Nova CDV (Nova Biomedical). Titer was measured at harvest using HPLC analysis. The transcript ratio was determined using ddPCR according to the manufacturer's recommendations (DROPLET DIGITAL™ PCR (ddPCR™) system (Bio Rad, Hercules, CA)). The molar ratio of long heavy chain to short heavy chain was determined by rCE-SDS.

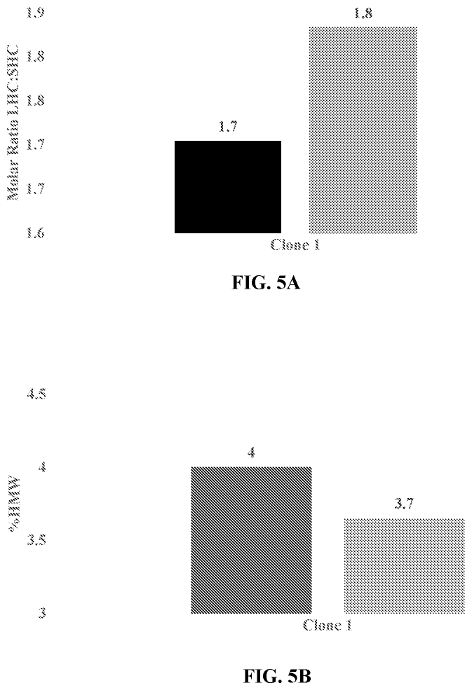

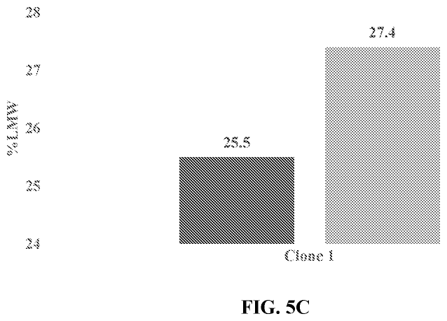

An increase of molar ratio of long heavy chain (LHC) to short heavy chain (SHC) was seen in the sample that was temperature shifted to 32.5° C. which indicated preferential expression of the long heavy chain relative to the short heavy chain at the lower temperature, FIG. 5A, 36° C. (black bar), 32.5° C. (gray bar). This correlated with a decrease of high molecular weight (% HMW) as determined by SEC in the sample that was cultured at 36° C. (FIG. 5B) and an increase of % LMW as determined by SEC in the temperature shifted sample, see FIG. 5C. The HMW impurities were formed by the short heavy chain and the LMW impurities were formed by the long heavy chain. The integral viable cell density was relatively unchanged and the titer was similar between the two culture conditions. The transcript ratio of long heavy chain to short heavy chain also increased at lower temperature.

### Mock Perfusion Culture

Two clones (Clone 2 and Clone 3) expressing Bispecific A were selected from a group of clones from the initial clonal cell screening and tested in a small-scale mock perfusion culture to study the transcript and protein response time versus temperature change in a culture process that mimics a high cell density perfusion culture in a bioreactor. The clones were cultured at a constant temperature (36° C.) and with a temperature shift (36° C. to 32.5° C.).

Two 24 deep well plates were inoculated with 200×10⁵ cells/ml/well for each of the clones. The plates were incubated at 36° C., 5% CO₂ on day 0. On day 1, the temperature was decreased to 32.5° C. for one plate from each clone while the second plate for each clone was kept at 36° C. The plates were harvested on day 3. A daily medium exchange using a serum-free, chemically defined medium was performed to remove spent medium and add fresh medium to mimic a perfusion culture. Analytical measurements were taken each day.

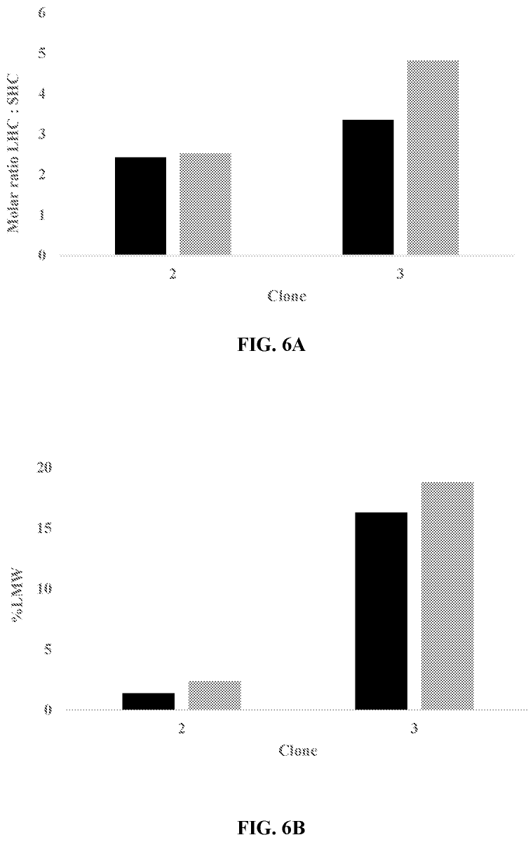

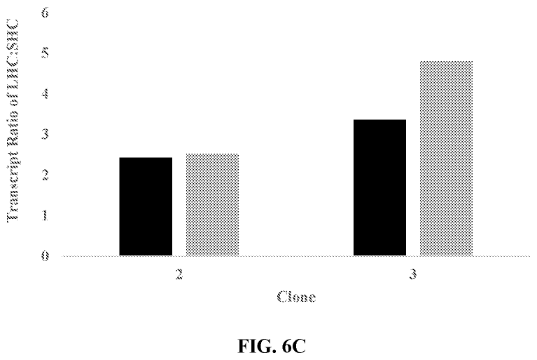

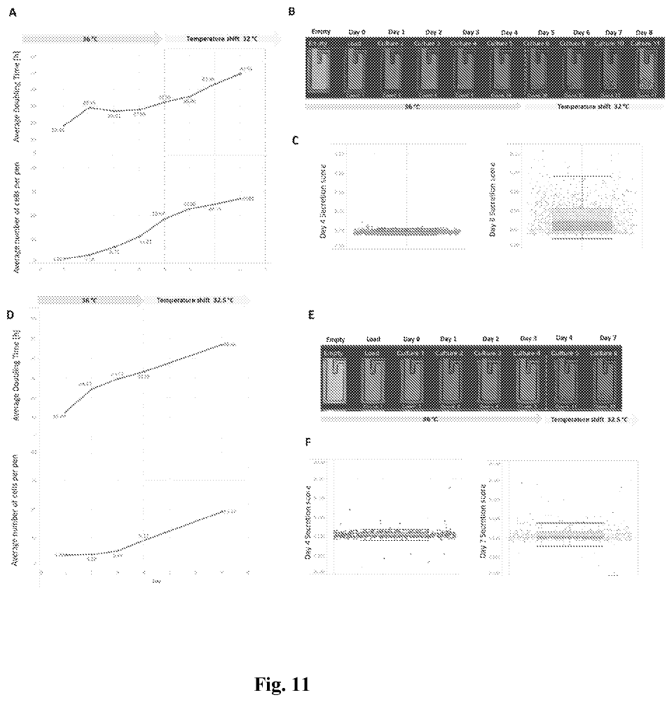

There was an increase of 9% (Clone 2) and 13% (Clone 3) in the molar ratio of long heavy chain (LHC) to short heavy chain (SHC) at low temperature at 24 hours post-temperature shift from 36° C. to 32.5° C. compared to the cultures held at a constant temperature of 36° C., FIG. 6A (36° C. black bar. 32.5° C. gray bar.). This indicated that there was preferential expression of the long heavy chain relative to the short heavy chain at the lower temperature. The increase in the molar ratio was consistent with increased % LMW which comprised long heavy chain homodimer and monomer impurities, FIG. 6B. The Transcript Ratio is seen in FIG. 11C. Both the cell growth and titer were lower for the clones subjected to the temperature shift.

As seen in Experiment 1, the molar ratio of the long heavy chain and a short heavy chain of Bispecific 1 was also found to be influenced by the temperature of the culture. An increase in the molar ratio of the long heavy chain to short heavy chain was consistent with the formation of increased impurities comprising the long heavy chain at lower temperatures.

### Example 3

This experiment looked at the effect of temperature during clone selection and the impact on product quality at a transcriptional level for an asymmetric bispecific antibody, Bispecific C. Bispecific C has two heavy chains, a “long heavy chain” having the longer amino acid sequence and a “short heavy chain” having the shorter amino acid sequence.

### Fed-Batch Shake Flask Culture

Three clones (Clone 1, Clone 2, and Clone 3) were selected from the initial clone screening of Bispecific B. A 10-day fed-batch temperature study was conducted in shake flasks, as described in Example 2 above, to determine the transcript and protein response of the clonal cells in a process at a constant temperature of 36° C. and in a process that included a temperature shift from 36° C. to 32.5° C.

One IL shake flask (Corning, Corning, NY) with 250ml working volume of serum free, chemically defined culture medium was initiated for each clone. Each flask was inoculated at 8×10⁵ cells/ml on day 0. The shake flasks were maintained at 36° C., 5% CO₂, 160 rpm, for seven days and fed on days 3 and 6, at 7% of post-inoculation volume.

On day 7, each flask was split into two flasks with a 60 ml working volume of serum free, chemically defined culture medium. One flask was maintained at 36° C., the second flask was maintained at 32.5° C. Both were cultured at 5% CO₂, agitation at 160 rpm, until day 10. The cultures were fed on day 8 at 7% of post-inoculation volume. Analytical measurements were taken throughout the 10-day culture.

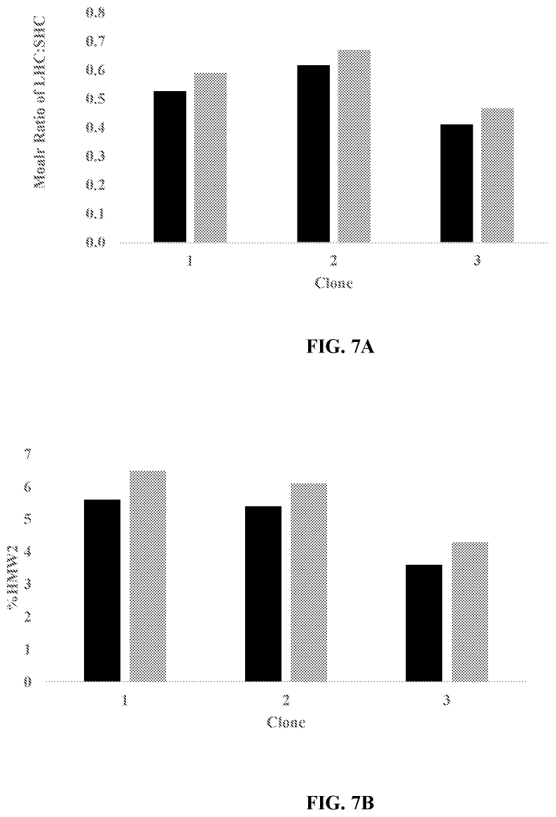

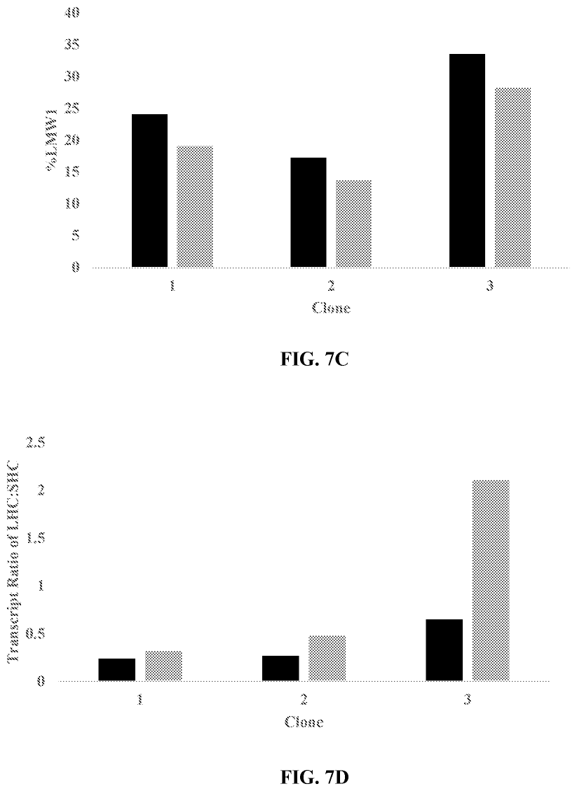

The molar ratio of the long heavy chain to the short heavy chain increased at the lower temperature which was an indication of preferential expression of the long heavy chain relative to the short heavy chain, see FIG. 7A (36° C. black bars, 32.5° C. gray bars.). This was consistent with increased % HMW2 which comprised long heavy chain homodimer impurities, (FIG. 7B) and decreased % LMW1 which comprised short heavy chain homodimer impurities, (FIG. 7C). The transcription ratio is seen in FIG. 7C. A similar titer was observed between the two temperature regimes. There were small differences (10%) in integral viable cell density (IVCD) between the two temperature regimes.

### Mock Perfusion Culture

Bispecific B clones 2 and 3 were also tested in a small-scale mock perfusion culture to study the transcript and protein response time versus temperature change in a culture process that mimics high cell density perfusion culture in a bioreactor, as described above.

Two 24 well deep well plates were inoculated with 200×10⁵ cells/ml/well for each of Clones 2 and 3 and incubated at 36° C., 5% CO₂ on day 0. On day 1, the temperature for one plate for each clone was decreased to 32.5° C. while the other plate was maintained at 36° C. On day 3 the plates were harvested. A serum-free, chemically defined medium was used for the culture. A daily medium exchange was performed to remove spent medium and add fresh medium. Analytical measurements were taken each day, as described in the Examples above.

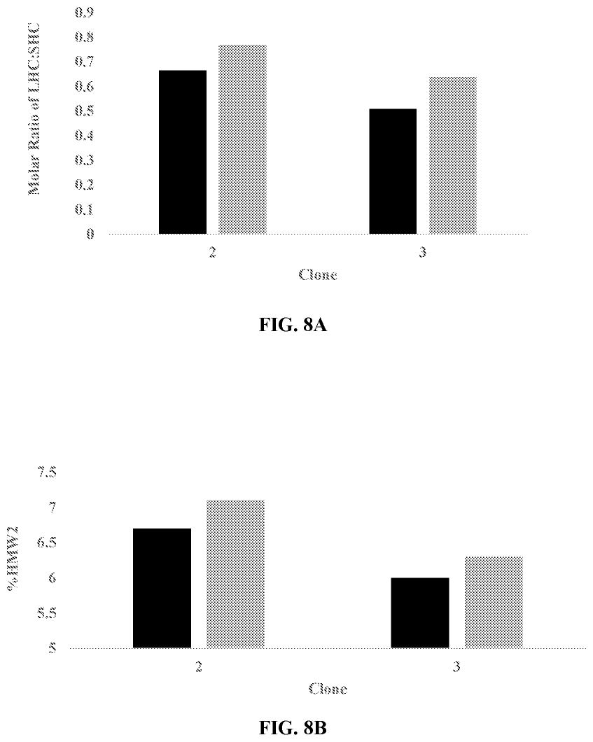

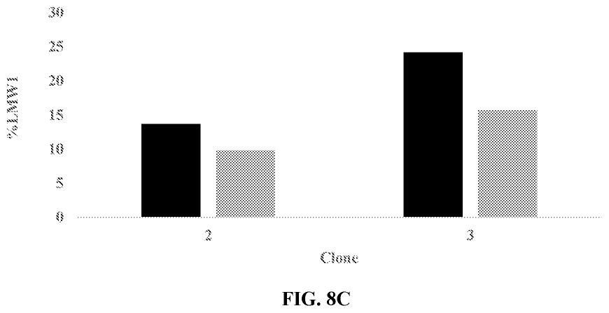

The molar ratio of long heavy chain to short heavy chain increased by 17% for Clone 2 and 25% for Clone 3 at 24 hours post-temperature shift from 36° C. to 32.5° C. compared to constant temperature at 36° C., See FIG. 8A (36° C. black bars, 32.5° C. gray bars.). This indicated preferential expression of the long heavy chain relative to the short heavy chain at the lower temperature. The increase in the molar ratio was consistent with increased % HMW2, as determined by SEC and decreased % LMW1, as determined by SEC, Sec FIGS. 8B and C. The HMW2 impurity comprised the long heavy chain homodimer and the LMW1 impurity comprised the short heavy chain homodimer. Slightly lower titer was seen at 32.5° C. for both clones on day 2.

As seen in Experiments 1 and 2, the molar ratio of the long heavy chain and a short heavy chain of Bispecific 2 was also found to be influenced by the temperature of the culture. An increase in the molar ratio of the long heavy chain to short heavy chain was consistent with the formation of increased impurities comprising the long heavy chain at lower temperatures.

As demonstrated in the experiments, the expression of the long heavy chain of asymmetric multispecific proteins was influenced by temperature, as shown by the increase in molar ratio of the expression of long heavy chain to short heavy chain at lower temperatures. The change in the ratio had an impact on the formation of product-related impurities which influenced the difficulty in separate them from the desired product. Impurities comprising the long heavy chain were found to be surprisingly more difficult to clear in downstream purification operations compared to impurities comprising the short heavy chain. Using temperature as a lever to balance the expression long and short heavy chains has a positive impact on the purification, activity, and product quality of asymmetric proteins.

### Example 4

These experiments provide a method to control cell growth on a nanofluidic chip through temperature shift to extend culture duration and measurement of protein secretion levels and to minimize the risks of cell cross contamination during export procedure executed under reduced temperature settings. Single cells expressing either a monoclonal or multispecific antibody were deposited on a nanofluidic chip and subjected to a static temperature culture mode where the same temperature was maintained for the duration of the culture or a biphasic culture mode where after an initial incubation at a first temperature condition, a temperature shift (lowering the temperature) was applied. The temperature reduction led to growth inhibition, preventing cell overgrowth therefore reducing cell-cross contamination risks and enabled long-term culture, while altering recombinant protein production profiles. The effects of static and shifted temperature on the recombinant protein productivity were analyzed and used to aid early clone selection.

CHO cell lines were generated by transfecting a CHO host with plasmid DNA encoding a monoclonal or multispecific antibody. Following transfection, stably expressing pool populations were generated through repeated passaging in selective medium, where MSX selection stringency was also applied, until the cells reached above 90% viability and maintained consistent doubling times.

Throughout the process, the cells were cultured in either, T-175 flasks (Corning, Corning, NY), or 50 mL spin tubes (TPP, Trasadingen, Switzerland) in selective growth media with MSX selection stringency at 36° C., 5% CO₂ and 85% humidity. Cells were maintained by passaging multiple times a week at a target seed density.

### Static Single Temperature Culture (Control)

A CHO cell line was single cell loaded on OptoSelect™ chips (Design 1750. Berkeley Lights. Emeryville. CA) using the Beacon Instrument (Berkeley Lights, Emeryville, CA). OptoElectroPositioning (OEP™), localized electric field gradient settings and scripts for loading and exporting cells were provided by the manufacturer. Cells were cultured on the OptoSelect™ chips at 36° C. for up to 6 days using proprietary growth media and manufacturer recommended settings. Repeated imaging and cell counting were performed using the integrated 4× microscope and camera on the Beacon instrument. Evidence of clonal derivation was achieved by image of a single cell in a NanoPen™ chamber (0.0481 mm² area, and 1.70 nL volume). Secretion assays were performed using the Spotlight™ Human Fc Assay (Berkeley Lights, Emeryville, CA) with supplied method scripts and assay analyzer software (Cell Analysis Suite, Build 30552). Selected pens were exported using OEP™ settings to move cells out of the chambers followed by flushing off the chip and collecting into a 96-well microtiter plate prefilled with proprietary single cell cloning media. Clonally derived cell lines were recovered after export through culture in 96-well plates (Corning, Corning, NY) which were maintained at 36° C. 5% CO₂, 85% relative humidity. The cells were monitored for growth through repeated imaging using CellMetric imaging system.

### Static Single Temperature Culture With Temperature Shift Implemented During Export Procedure

A CHO cell line was single cell loaded on OptoSelect™ chips (Design 1750, Berkeley Lights, Emeryville, CA) using the Beacon Instrument (Berkeley Lights, Emeryville, CA). OptoElectroPositioning settings and scripts for loading and exporting cells were provided by the manufacturer. Cells were cultured on the OptoSelect™ chips at 36° C. for up to day 4 using proprietary growth media and manufacturer recommended settings. On day 4 during export procedure the temperature was shifted to 32° C. and maintained until the end of export procedure. A static culture at 36° C. was maintained for the entire 4-day culture period. Scripts for exporting were provided by the manufacturer and modified where the export procedure was executed with temperature shift settings at 32° C. Repeated imaging and cell counting were performed using the integrated 4X microscope and camera on the Beacon instrument. Evidence of clonal derivation was achieved by image of a single cell in a NanoPen™ chamber (0.0481 mm² area, and 1.70 nL volume). Secretion assays were performed using the Spotlight™ Human Fc Assay (Berkeley Lights, Emeryville, CA) with supplied method scripts and assay analyzer software (Cell Analysis Suite, Build 30552). Selected chambers were exported using OEP™ settings to move cells out of pens followed by flushing off the chip and collecting the cells into a 96-well microtiter plate prefilled with proprietary single cell cloning media.

### Temperature Shift Culture

A CHO cell line was single cell loaded on OptoSelect™ chips (Design 1750, Berkeley Lights, Emeryville, CA) using the Beacon Instrument (Berkeley Lights, Emeryville, CA). OptoElectroPositioning settings and scripts for loading and exporting cells were provided by the manufacturer Cells were cultured on the OptoSelect™ chips at 36° C. until Day 3, 4 or 5 (as indicated) using proprietary growth media and manufacturer recommended settings. A temperature shift to 32° C. or 32.5° C. was applied on Day 3, 4 or 5 and the culture was maintained at the lower temperature until Day 7 or 8. Scripts for exporting were provided by the manufacturer where the export procedure was executed at either manufacturer recommended settings or with temperature shift settings of 32° C. Repeated imaging and cell counting were performed using the integrated 4× microscope and camera on the Beacon instrument. Evidence of clonal derivation was achieved by image of a single cell in a NanoPen™ chamber (0.0481 mm² area, and 1.70 nL volume). Secretion assays were performed using the Spotlight™ Human Fc Assay (Berkeley Lights, Emeryville, CA) with supplied method scripts and assay analyzer software (Cell Analysis Suite, Build 30552). Selected chambers were exported using OEP™ to move cells out of chambers followed by flushing off the chip and collecting the cells into a 96-well microtiter plate prefilled with proprietary single cell cloning media.

### Results

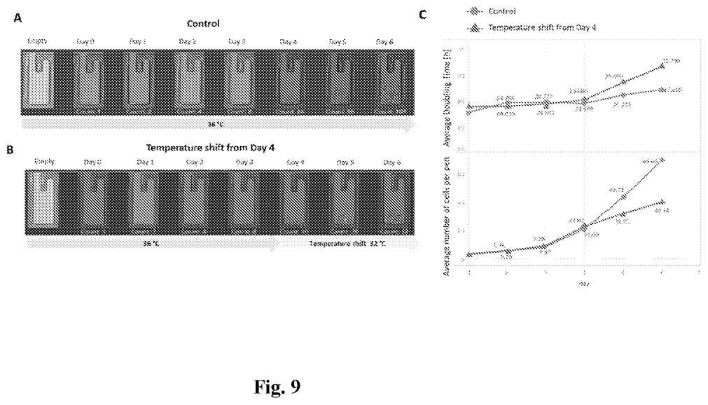

FIG. 9 shows that temperature shift implemented during on chip culture results in growth inhibition and allows for long-term culture. (A) Representative brightfield NanoPen™ chamber images collected throughout experiment duration on Berkeley Lights Bacon platform, where single cells expressing monoclonal antibody were loaded on a chip into individual chambers and the chip was cultured for 6 days at 36° C. The cell counts generated by the instrument software are displayed below each timepoint. Cell population on day 6 occupied the majority of the chamber content and expanded close to the neck area of the chamber, preventing reliable secretion assessment through Spotlight™ Human Fc assay and creating high risk of cross contamination during potential export procedure. (B) Representative brightfield chamber images collected throughout experiment duration on Berkeley Lights Bacon platform, where single cells expressing monoclonal antibody were loaded on a chip into individual chambers and the chip was cultured for 3 days at 36° C. and temperature was subsequently reduced to 32° C. for the remaining experiment duration. The cell counts generated by the instrument software are displayed below each timepoint. Temperature shift implemented from Day 4 resulted in growth inhibition and prevented cell expanding above the maximum recommended height of the chamber. (C) Average doubling time (top panel) and average number of cells per chamber (bottom panel) measured on each day throughout experiment duration. Implementing temperature shift from Day 4 resulted in growth inhibition manifested by increased average doubling time and decreased average cell counts when compared to the control condition.

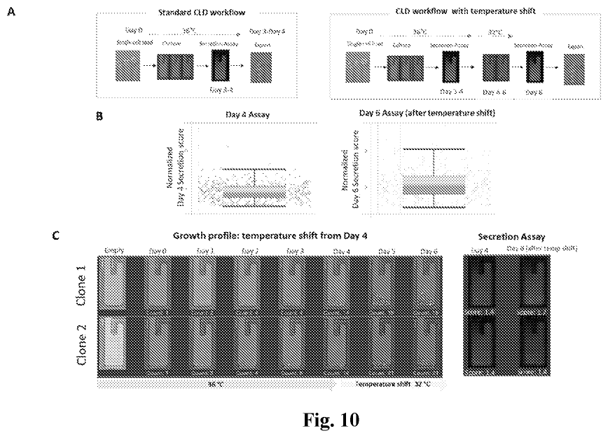

FIG. 10 Shows that implementing temperature shift during a standard cell line development (CLD) workflow on the Berkeley Light Beacon platform alters monoclonal antibody production profiles. (A) Schematic representation of standard Cell Line Development cloning workflow on the Beacon platform (left panel) and Cell Line Development cloning workflow with temperature shift (right panel) implemented in B and C. (B) Normalized secretion score data corresponding to Spotlight™ Human Fc assay conducted before temperature shift on Day 4 (left panel) and after temperature shift on Day 6 (right panel). Cell secretion profiles measured after temperature shift showed more diverse distribution allowing better distinction of highly producing clones form poorly secreting cell lines. (C) Brightfield and Spotlight™ Human Fc Assay fluorescent images collected throughout experiment duration on Berkeley Lights Bacon platform corresponding to two clonally derived cell lines expressing monoclonal antibody where CLD workflow with temperature shift was implemented. The cell counts generated by the instrument software and normalized Secretion score are displayed below each timepoint. Incubation in decreased temperature conditions resulted in growth arrest for both clones. While both cell lines demonstrated comparable secretion profiles on Day 4 of the workflow. Clone 1 but not Clone 2 displayed increased secretion levels in response to temperature shift.

FIG. 11 Shows that implementing temperature shift during CLD workflow on the Berkeley Light Beacon platform achieves growth inhibition and alters recombinant protein production profiles for cell lines expressing IgG4 monoclonal antibodies and multispecific antibody formats. (A)(D) Average doubling time (top panel) and average number of cells per chamber (bottom panel) measured on each day throughout experiment duration for clonally derived cell lines expressing IgG4 monoclonal antibody and multispecific antibody, respectively. Temperature settings for each timepoint of the experiment are outlined on the top of the graph. Implementing temperature shift from Day 4 or Day 3, respectively, resulted in growth inhibition measured by increased doubling time and decreased cell counts for both IgG4 and multispecific antibody expressing cell lines. (B)(E) Representative brightfield chamber images collected throughout experiment duration on Berkeley Lights Bacon platform of clonally derived cell lines expressing monoclonal antibody or multispecific antibody, respectively. The cell counts generated by the instrument software are displayed below each timepoint. (C)(F) Spotlight™ Human Fc Assay score data corresponding to Day 4 and Day 8 or Day 7 timepoints, respectively. Secretion profiles achieved after prolonged incubation in decreased temperature showed more diverse distribution allowing better distinction of highly producing clones form poorly secreting cell lines.

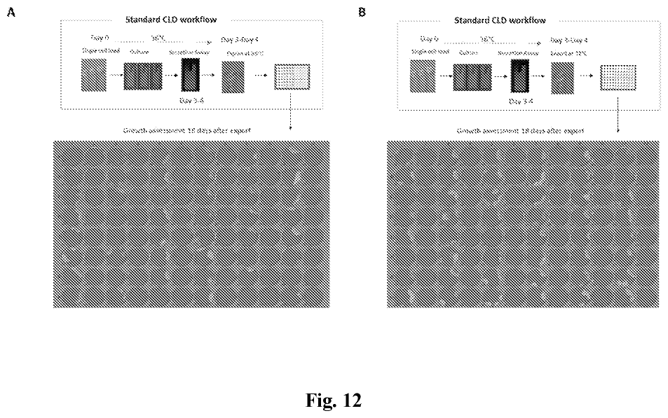

FIG. 12 Shows that export procedure conducted at 32° C. temperature results in similar clone recovery when compared to export executed at 36° C. per standard CLD workflow. (A)(B) Top panel: schematic representation of standard Cell Line Development cloning workflow on the Beacon platform and Cell Line Development cloning workflow with temperature shift implemented during export process, respectively. Bottom panel: representative images of 96 well export plates acquired 18 days after export procedure showing comparable clone recovery efficiency regardless of export temperature implemented at the single cell cloning stage.

### CONCLUSION

We explored alternative Cell Line Development workflow conditions on Berkeley Lights technology platform to better control for cell growth, minimize risks associated with cell cross-contamination during the export procedure, achieve higher secretion levels and improve early clone selection. We demonstrated that temperature shift introduced after Day 3 during CLD workflow results if lower growth and extended cell doubling times which enabled long term culture. Growth inhibition at lower temperatures can be leveraged during export procedure as dividing cells that may reside near chamber entry/exit can potentially escape into the channel area and cross-contaminate fluidic paths and consequently compromise the export purity. We provide evidence that implementing lower temperature during export process does not impact clone recovery during clone expansion at 96 well plate stage. Furthermore, implementing temperature shift allows to conduct secretion assays at later timepoints, and better discriminate between highly expressing clones and eliminate poorly performing cell lines. Finally, our data indicates that culturing under lower temperatures can alter recombinant protein secretion profiles that can potentially be leveraged for identifying clones with unique phenotypes.

What is claimed is:

1. A method for modulating the product quality of a recombinant asymmetric multispecific antibody expressed by a cell during cell culture comprising the steps:
a) establishing at least two cell cultures each inoculated with the same cell line expressing the asymmetric multispecific antibody;
b) culturing at least one cell culture at a first temperature regime that consists of a single temperature and at least one cell culture at a second temperature regime;
c) comparing at least one product-related impurity in the cell culture at each temperature regime;
d) selecting the temperature regime that reduces the expression of at least one product-related impurity comprising an unpaired or mis-paired long heavy chain;
e) culturing the cell line at the selected temperature regime; and
f) harvesting the recombinant asymmetric multispecific antibody.

2. The method according to claim 1, wherein the first temperature regime is selected from 36° C. to 37° C.

3. The method according to claim 1, wherein the second temperature regime comprises a temperature shift from a first temperature to a second temperature that is higher or lower than the first temperature.

4. The method according to claim 3, wherein the second temperature is selected from 28° C. to 35° C.

5. The method according to claim 3, wherein the second temperature is about 1° C. to about 9° C. lower than the first temperature.

6. The method according to claim 1, wherein the first temperature regime is a single temperature from 36° C. to 37° C. and the second temperature regime comprises at least one temperature shift from a first temperature of 36° C. to 37° C. to a second temperature of 28° C. to 35° C.

7. The method according to claim 1, wherein the product-related impurity results from an imbalance in the ratio of long heavy chain to short heavy chain.

8. The method according to claim 1, wherein the product-related impurity comprises an unpaired or mis-paired long heavy chain.

9. The method according to claim 8, wherein the product-related impurity comprises an unpaired or mis-paired long heavy chain and is selected from a homodimer, half antibody, protein aggregate, antibody fragment, combination of antibody fragments, and unpaired antibody fragments.

10. The method according to claim 1, wherein the cell culture temperature regime may be further selected for modulation of the amount of expression, productivity, growth, yield, and/or other desired product quality attributes of the asymmetric multispecific antibody.

11. The method according to claim 1, wherein the asymmetric multispecific antibody comprises a mutein.

12. The method according to claim 1, wherein the asymmetric multispecific antibody is an asymmetric multispecific anti-PD-1 antibody comprising a single IL-21 mutein attached to one of the two antibody heavy chains of the anti-PD-1 antibody.

13. The method according to claim 7, wherein the long heavy chain comprises an anti-PD-1 antibody heavy chain having an attached IL-21 mutein.

14. A method for modulating the product quality of a recombinant asymmetric multispecific antibody comprising a mutein expressed by a cell during cell culture comprising the steps:
a) establishing at least two cell cultures each inoculated with the same cell line expressing the asymmetric multispecific antibody;
b) culturing at least one cell culture at a first temperature regime that consists of a single temperature and at least one cell culture at a second temperature regime;
c) comparing at least one product-related impurity comprising an unpaired or mis-paired long heavy chain produced by a cell culture at each temperature regime; and
d) selecting the temperature regime that reduces the expression of at least one product-related impurity comprising an unpaired or mis-paired long heavy chain.

15. The method according to claim 14, wherein the mutein is an IL-21 mutein.

16. The method according to claim 15, wherein the IL-21 mutein comprises amino acid substitutions at any two of positions 5, 9, 73, and 76 of SEQ ID NO: 1; wherein said amino acid substitutions are selected from: A, E, or Q at position 5, E or A at position 9, A or Q at position 73, and A, D, or E at position 76.

17. The method according to claim 15, wherein the IL-21 mutein comprises the amino acid sequence of any of SEQ ID NOs: 233-245.

18. The method according to claim 14, wherein the asymmetric multispecific antibody comprises a mutein attached to the C-terminus of one of the two antibody heavy chains of an anti-PD-1 antibody.

19. The method according to claim 18, wherein the anti-PD-1 antibody comprising:
two light chains, each comprising a LC CDR1, LC CDR2, and LC CDR3 comprising the amino acid sequence of SEQ ID NOs: 385, 386, and 387, respectively; and
two heavy chains, each comprising a HC CDR1, LC CDR2, and LC CDR3 comprising the amino acid sequence of SEQ ID NOs: 382, 383, and 384, respectively.

20. The method according to claim 18, wherein the anti-PD-1 antibody comprises two light chains comprising the amino acid sequence of SEQ ID NO: 389 and two heavy chains comprising the amino acid sequence of SEQ ID NO: 388.

21. The method according to claim 18, wherein the anti-PD-1 antibody comprises:
two light chains, each comprising a LC CDR1, LC CDR2, and LC CDR3 comprising the amino acid sequence of SEQ ID NOs: 365, 366, and 367, respectively; and
two heavy chains, each comprising a HC CDR1, HC CDR2, and HC CDR3 comprising the amino acid sequence of SEQ ID NOs: 362, 363, and 364, respectively.

22. The method according to claim 18, wherein the anti-PD-1 antibody comprises two light chains comprising the amino acid sequence of SEQ ID NO: 369 and two heavy chains comprising the amino acid sequence of SEQ ID NO: 368.

23. The method according to claim 18, wherein the anti-PD-1 antibody comprises:
(i) two light chains, each comprising the amino acid sequence of SEQ ID NO: 391; a heavy chain attached to an IL-21 mutein comprising the amino acid sequence of any one of SEQ ID NOs: 501-506; and a heavy chain comprising the amino acid sequence of any one of SEQ ID NOs: 556-558; or
(ii) two light chains, each comprising the amino acid sequence of SEQ ID NO: 371; a heavy chain attached to a single IL-21 mutein comprising an amino acid sequence of any one of SEQ ID NOs: 513-518; and a heavy chain comprising the amino acid sequence of any one of SEQ ID NOs: 559-561.

24. The method according to claim 18, wherein the anti-PD-1 antibody comprises:
two light chains, each comprising the amino acid sequence of SEQ ID NO: 391;
one heavy chain comprising the amino acid sequence of SEQ ID NO: 556; and
one heavy chain attached to an IL-21 mutein comprising the amino acid sequence of SEQ ID NO: 501.

25. The method according to claim 18, wherein the anti-PD-1 antibody comprises:
two light chains, each comprising the amino acid sequence of SEQ ID NO: 371;
one heavy chain comprising the amino acid sequence of SEQ ID NO: 559; and
one heavy chain attached to an IL-21 mutein comprising the amino acid sequence of SEQ ID NO: 513.

26. The method according to claim 14, wherein the asymmetric multispecific antibody comprises an IL-21 mutein attached to the C-terminus of one of the two antibody heavy chains of an anti-PD-1 antibody.

27. The method according to claim 14, wherein the long heavy chain comprises an anti-PD-1 antibody heavy chain having an attached IL-21 mutein.

28. A method for modulating the product quality of a conjugate of an IL-21 mutein linked to the C-terminus of one of the two antibody heavy chains of an anti-PD-1 antibody expressed by a cell during cell culture comprising the steps:
a) establishing a cell culture inoculated with a cell line expressing the antibody;
b) culturing the cells at 36+1° C. for the duration of the culture; and
c) harvesting the antibody;
wherein amount of at least one product-related impurity resulting from an imbalance in the ratio of long heavy chain to short heavy chain in the harvested cell culture is decreased compared to the amount of the same product-related impurity in the harvest from a cell culture exposed to a temperature of 32° C. to 34° C. at some point during the culture.

29. A method for selecting a cell line expressing an asymmetric multispecific antibody comprising a mutein comprising the steps:
a) establishing at least one clonally derived cell line expressing the antibody;
b) establishing at least two cell cultures from one or more of the clonally derived cell lines, wherein at least one cell culture from each cell line is cultured at a first temperature regime and at least one cell culture from each cell line is cultured at a second temperature regime;
c) comparing the expression of at least one product-related impurity comprising an unpaired or mis-paired long heavy chain produced by the cells cultured at each temperature regime; and
d) selecting a cell line that resulted in reduced expression of at least one product-related impurity comprising an unpaired or mis-paired long heavy chain.

30. The method of claim 29, wherein the selected cell line is then cultured at the temperature regime that resulted in reduced expression of at least one product-related impurity comprising an unpaired or mis-paired long heavy chain.

31. The method according to claim 29, wherein the first temperature regime is selected from about 36° C. to about 37° C.

32. The method according to claim 29, wherein the second temperature regime comprises a temperature shift from a first temperature to a second temperature that is higher or lower than the first temperature.

33. The method according to claim 32, wherein the second temperature is selected from about 28° C. to about 35° C.

34. The method according to claim 29, wherein the second temperature is about 1° C. to 9® C. lower than the first temperature.

35. The method according to claim 29, wherein the first temperature regime is a single temperature from 36° C. to 37° C. and the second temperature regime comprises at least one temperature shift from a first temperature of 36° C. to 37° C. to a second temperature of 28° C. to 35° C.

36. The method according to claim 29, wherein the product-related impurity comprising an unpaired or mis-paired long heavy chain results from an imbalance in the ratio of long heavy chain to short heavy chain.

37. The method according to claim 35, wherein the product-related impurity comprising an unpaired or mis-paired long heavy chain results from an increase in the ratio of the long heavy chain to the short heavy chain.

38. The method according to claim 29, wherein the product-related impurity comprising an unpaired or mis-paired long heavy chain and is selected from a homodimer, half antibody, protein aggregate, antibody fragment, combination of antibody fragments, and unpaired antibody fragments.

39. The method according to claim 29, wherein the cell culture temperature regime may be further selected for a temperature regime that also modulates the amount of expression, productivity, growth, and/or other desired product quality attributes of the asymmetric multispecific antibody.

40. The method according to claim 29, wherein the asymmetric multispecific antibody is an asymmetric multispecific anti-PD-1 antibody comprising a single IL-21 mutein attached to one of the two antibody heavy chains of the anti-PD-1 antibody.

41. The method according to claim 29, wherein the long heavy chain comprises an anti-PD-1 antibody heavy chain having an attached IL-21 mutein.

42. A method for modulating production of at least one recombinant asymmetric multispecific antibody product-related impurity comprising an antibody heavy chain having an attached IL-21 mutein during cell culture through cell culture temperature comprising the steps:
a) selecting the temperature regime that reduces the expression of at least one product-related impurity comprising an unpaired or mis-paired long heavy chain comprising a long heavy chain;
b) culturing the cell line at the selected temperature regime; and
c) harvesting the recombinant asymmetric multispecific antibody.

43. The method according to claim 42, wherein the asymmetric multispecific antibody is an asymmetric multispecific anti-PD-1 antibody comprising a single IL-21 mutein attached to one of the two antibody heavy chains of the anti-PD-1 antibody.

44. The method according to claim 42, wherein the long heavy chain comprises an anti-PD-1 antibody heavy chain having an attached IL-21 mutein.

45. A method for producing an isolated, purified, recombinant asymmetric multispecific antibody, the method comprising the steps:
a) isolating at least one single stably transformed cell expressing the asymmetric multispecific antibody and establishing a clonally derived culture;
b) establishing at least two cell cultures from one or more of the clonally derived cultures;
c) culturing at least one cell culture from each clonally derived culture at a first temperature regime comprising a single temperature and culturing at least one cell culture from each clonally derived culture at a second temperature regime;
d) comparing the amount of at least one product-related impurity comprising an unpaired or mis-paired long heavy chain produced by the cells cultured at each temperature regime;
e) selecting the cell culture that modulated production of the product related impurity;
f) establishing a cell line expressing the asymmetric multispecific antibody from the selected cell culture;
g) inoculating a bioreactor with the cell line expressing the asymmetric multispecific antibody;
h) culturing the cells to express the asymmetric multispecific antibody at the temperature regime that modulated the production of the product-related impurity;
i) harvesting the recombinant asymmetric multispecific antibody from the cell culture;
j) processing the recombinant multispecific antibody through one or more chromatography unit operations; and
k) obtaining an isolated, purified, recombinant asymmetric multispecific antibody.

46. An isolated, purified, recombinant multispecific antibody according to claim 45.

47. A pharmaceutical composition comprising the isolated, purified, recombinant multispecific antibody according to claim 45.

48. The method according to claim 45, wherein the asymmetric multispecific antibody is an asymmetric multispecific anti-PD-1 antibody comprising a single IL-21 mutein attached to one of the two antibody heavy chains of the anti-PD-1 antibody.

49. The method according to claim 48, wherein the long heavy chain comprises an anti-PD-1 antibody heavy chain having an attached IL-21 mutein.

50. A method for controlling the growth of cells in a nanofluidic chamber of a nanofluidic chip comprising the steps of:
(a) isolating a single cell in to a nanofluidic chamber of a nanofluidic chip, wherein said cell comprises an expression construct capable of expressing a recombinant protein;
(b) culturing the cell at a first temperature;
(c) at a predetermined point, culturing the cells at a second temperature; and
(d) exporting the cells out of the nanofluidic chamber and into a culture vessel.

51. The method according to claim 50, wherein the number of cells per chamber at export is lower compared to a single cell cultured under similar conditions at a constant temperature for the duration of the culture.

52. The method according to claim 50, wherein the first temperature is selected from 35° C. to 37° C.

53. The method according to claim 50, wherein the second temperature is selected from 28° C. to 34° C.

54. The method according to claim 50, wherein the second temperature is about 1° C. to about 9° C. lower than the first temperature.

55. The method according to claim 50, wherein the first temperature is 36° C. and the second temperature is 32° C. to 32° C.

56. The method according to claim 50, wherein the predetermined point is on day 3 to day 5 of the culture.

57. The method according to claim 50, wherein export is on day 4 to day 8 of the culture.

58. The method according to claim 50, wherein the nanofluidic chip comprises 1758 chambers, 3,500 chambers, 11,000 chambers, 14,000 chamber, or 20,000 chambers.

59. The method according to claim 50, wherein the culture vessel is a multi-well plate.

60. A method for minimizing cell cross contamination during export of cells grown in a nanofluidic chamber of a nanofluidic chip comprising the steps of:
(a) isolating a single cell in to a nanofluidic chamber of a nanofluidic chip, wherein said cell comprises an expression construct capable of expressing a recombinant protein;
(b) culturing the cell at a first temperature;
(c) at a predetermined point, culturing the cells at a second temperature; and
(d) exporting the cells out of the nanofluidic chamber and into a culture vessel;
wherein the number of cells per chamber at export is lower compared to a single cell cultured under similar conditions a constant temperature for the duration of the culture.

61. A method to improve clone selection for cells grown in a nanofluidic chamber of a nanofluidic chip comprising the steps of:
(a) isolating a single cell in to a nanofluidic chamber of a nanofluidic chip, wherein said cell comprises an expression construct capable of expressing a recombinant protein;
(b) culturing the cell at a first temperature;
(c) lowering the temperature of the culture to a second temperature no earlier than day 3 of the culture; and
(d) exporting the cells out of the nanofluidic chamber at least one to four days after the temperature shift.

62. The method according to claim 61, wherein the protein secretion profiles measured before and after the temperature shift are compared.
